(19) United States
(12) Patent Application Publication
(10) Pub. No.: US 2025/0034635 A1
(43) Pub. Date: Jan. 30, 2025

(54) METHODS AND COMPOSITIONS FOR DUAL NANOPORE SEQUENCING

(71) Applicant: ELECTRONIC BIOSCIENCES, INC., San Diego, CA (US)

(72) Inventors: John Andrew WISNIEWSKI, South Sale Lake, UT (US); Vanja PANIC, Salt Lake City, UT (US); Eric Mayo PETERSON; Aaron FLEMING, Midvale, UT (US); Eric Nathan ERVIN, Salt Lake City, UT (US)

(21) Appl. No.: 18/710,411

(22) PCT Filed: Nov. 15, 2022
(86) PCT No.: PCT/US2022/049980

§ 371 Date: May 15, 2024

Related U.S. Application Data
(60) Provisional application No. 63/279,599, filed on Nov. 15, 2021.

(51) Int. Cl. C12Q 1/6869; C12Q 1/6806; G01N 33/487

(52) U.S. Cl. CPC C12Q 1/6869; C12Q 1/6806; G01N 33/48721

(57) ABSTRACT

Methods and compositions are provided for making and using capture tags attached to a target polymer in a dual nanopore sequencing system. In alternative embodiments, provided are systems, and products of manufacture and kits have contained therein, or comprise, a system as provided herein, wherein the system can comprise: a dual nanopore device; and a tagged target polymer comprising a capture tag at a first distal end and a capture tag at a second distal end, wherein the capture tag comprises a single-stranded tail.

### RELATED PATENT APPLICATION

This patent application claims the benefit of U.S. Provisional Patent Application No. 63/279,599 filed Nov. 15, 2021, entitled “METHODS AND COMPOSITIONS FOR DUAL NANOPORE SEQUENCING”, naming John Wisniewski, Vanja Panic, Eric Peterson, Aaron Fleming and Eric N. Ervin as inventors. The entire content of the foregoing patent application is incorporated herein by reference for all purposes, including all text, tables and drawings.

### FIELD

The technology relates in part to use of nanopore devices, such as for sequencing nucleic acids, for example. In alternative embodiments, provided are systems, and products of manufacture and kits have contained therein, or comprise, a system as provided herein, wherein the system can comprise: a dual nanopore device; and a tagged target polymer comprising a capture tag at a first distal end and a capture tag at a second distal end, wherein the capture tag comprises a single-stranded tail.

### BACKGROUND

For a nanopore reader to sequence a polymer molecule, i.e., a nucleic acid, protein, peptide, polymer, etc., the molecule needs to be translocated from the bulk solution in which it is contained to the mouth or opening aperture of the nanopore, followed by threading of one of the polymer's ends into the nanopore. Translocating from bulk solution and then threading into and through a nanopore typically occurs via diffusion, electrophoresis, electroosmosis and/or pressure driven flow. To enable sequencing, the diameter of the nanopore will be narrow enough to ensure that the polymer is fully extended, in a primary structural state, rather than in a double-stranded, folded, balled up, secondary or tertiary structural state.

### SUMMARY

Provided herein in certain aspects are systems, methods and compositions for modifying and capturing long single-stranded nucleic acids or an individual strand from double-stranded DNA or an RNA/DNA heteroduplex, as well as proteins, peptides, and other polymers across a dual nanopore sequencing device, such that these entities can then be controllably processed and sequenced.

Provided herein, in certain aspects are methods for associating, joining or linking a target polymer with dual nanopores. Providing a target polymer comprising a first distal end, a second distal end, a capture tag at the first distal end and a capture tag at the second distal end, thereby providing a tagged polymer. Providing a first nanopore and a second nanopore driving the first distal end of the tagged polymer through the first nanopore. Capturing the first distal end of the tagged polymer by the first nanopore. Driving the second distal end of the tagged polymer through the second nanopore. Capturing the second distal end of the tagged polymer by the second nanopore.

Also provided in certain aspects are methods for electrophoretically and/or electroosmotically driving at least a portion of a tagged polymer through a first nanopore or at least a portion of a tagged polymer through a second nanopore, identifying monomeric units of the tagged polymer as the tagged polymer translocates through the first nanopore or the second nanopore and determining the sequence of at least a portion of the target polymer.

Also provided in certain aspects are methods for sequencing a target polymer comprising (a) providing a target polymer comprising monomeric units, a first distal end, a second distal end, a capture tag at the first distal end and a capture tag at the second distal end, thereby providing a tagged polymer; (b) providing a first nanopore and a second nanopore; (c) driving the first distal end of the tagged polymer through the first nanopore, thereby capturing the first distal end of the tagged polymer by the first nanopore; (d) driving the second distal end of the tagged polymer through the second nanopore, thereby capturing the second distal end of the tagged polymer by the second nanopore; (e) electrophoretically and/or electroosmotically driving at least a portion of the tagged polymer through the first nanopore; (f) identifying monomeric units of the tagged polymer as the tagged polymer translocates through the first nanopore, thereby determining the sequence of at least a portion of the target polymer; (g) reversing the voltage bias between the first nanopore and the second nanopore; (h) electrophoretically and/or electroosmotically driving at least a portion of the tagged polymer through the second nanopore; (i) identifying monomeric units of the tagged polymer as the tagged polymer translocates through the second nanopore, thereby determining the sequence of at least a portion of the target polymer; (j) reversing the voltage bias between the first nanopore and the second nanopore; (k) electrophoretically and/or electroosmotically driving at least a portion of the tagged polymer through the first nanopore; (l) identifying monomeric units of the tagged polymer as the tagged polymer translocates through the first nanopore, thereby determining the sequence of at least a portion of the target polymer; and (m) repeating steps (g) to (l) to re-read the sequence or portion of the sequence of the target polymer.

Also provided in certain aspects is a capture tag comprising a double-stranded DNA segment with a single-stranded tail attached to a capture strand of the double-stranded DNA segment at a first end of the double-stranded DNA segment and the capture strand is configured to attach to a target strand of a nucleic acid target polymer at the second end of the double-stranded DNA segment. Also provided in certain aspects, is double-stranded DNA segment of a capture tag further comprising a complementary strand and at the first end of the double-stranded DNA segment the complementary strand comprises a non-complementary overhang or a single-stranded tail attached to a blocking molecule.

Also provided in certain aspects is a capture tag comprising a double-stranded DNA segment comprising a capture strand with a single-stranded tail attached at one end of the capture strand and the opposite end of the capture strand is configured to attach to a N terminus or a C terminus of a protein or peptide.

Also provided in certain aspects is a capture tag comprising a double-stranded DNA segment comprising a first end, a second end, a capture strand and a complementary strand. At the first end of the double-stranded DNA segment a single-stranded tail extends from the capture strand of the double-stranded DNA segment and a non-complementary overhang or a single-stranded tail extends from the complementary strand of the double-stranded DNA segment or there is no extension from the complementary strand of the double-stranded DNA segment. At the second end of the double-stranded segment a single-stranded tail extends from the capture strand of the double-stranded DNA segment or there is no extension from the capture strand of the double-stranded DNA segment, the capture strand is configured to attach to an N terminus or a C terminus of a protein or a peptide and a non-complementary overhang or a single-stranded tail extends from the complementary strand of the double-stranded DNA segment or there is no extension from the complementary strand of the double-stranded DNA segment.

Also provided in certain aspects is a capture tag comprising a rigid polymer comprising a single-stranded tail attached at a distal end and the opposite distal end is configured to attach to a N-terminus or a C-terminus of a protein or peptide.

Also provided in certain aspects are compositions comprising a target polymer comprising monomeric units, a first distal end and a second distal end, a capture tag at the first distal end and a capture tag at the second distal end of the target polymer.

In certain aspects the target polymer is a single-stranded RNA, a single-stranded DNA, a double-stranded DNA, a DNA/RNA hybrid, a protein, or a peptide.

Also provided in certain aspects are methods for providing target polymers with capture tags. In certain aspects the target polymer is a single-stranded RNA, a single-stranded DNA, a double-stranded DNA, an RNA/DNA heteroduplex, a protein, or a peptide.

Also provided in certain aspects are methods for sequencing target polymers comprising providing a target polymer with capture tags, providing a dual nanopore device, contacting the tagged target polymer and the dual nanopore device; and identifying monomeric units of the tagged target polymer as the tagged polymer translocates through the first nanopore or the second nanopore, thereby determining the sequence of at least a portion of the target polymer.

Also provided in certain aspects are systems comprising a dual nanopore device and a tagged polymer comprising a target polymer comprising monomeric units, a first distal end, a second distal end, a capture tag at the first distal end and a capture tag at the second distal end of the target polymer.

Also provided in certain aspects are methods of associating each of a plurality of target polymers with a dual nanopore device comprising providing a plurality of target polymers with capture tags, providing a plurality of dual nanopore devices, and contacting the plurality of tagged target polymers and the plurality of dual nanopore devices.

Also provided in certain aspects is a system comprising a plurality of dual nanopore devices and a plurality of tagged target polymers each tagged target polymer comprising monomeric units, a first distal end, a second distal end, a capture tag at the first distal end and a capture tag at the second distal end of the tagged target polymer.

Also provided in certain aspects are methods for sequencing a plurality of target polymers comprising providing a plurality of tagged target polymers, each comprising a target polymer with capture tags, providing a plurality of dual nanopore devices, contacting the plurality of tagged target polymers and the plurality of dual nanopore devices; and identifying monomeric units of the tagged target polymers as each of the tagged target polymers translocates through a dual nanopore device.

Certain implementations are described further in the following description, examples, claims and drawings.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIG. 1 illustrates an exemplary method for capturing one strand of a double-stranded DNA strand across a dual biological nanopore sequencing system, by placing poly T single-stranded DNA tails onto one of the strands of the double-stranded DNA target.

FIG. 2 illustrates an exemplary modification of long single-stranded DNA (ssDNA) or long single-stranded RNA (ssRNA) to improve capture frequency across a dual nanopore sequencing system; and “n” can be an integer between about 1 and 100 (AAAAAA is SEQ ID NO:1), as discussed in further detail, below.

FIG. 3A-B illustrates an exemplary method of making ssDNA more rigid with a longer persistence length. FIG. 3A shows conversion to double-stranded DNA. FIG. 3B shows conversion to a “hybrid double strand.”

FIG. 4 illustrates an exemplary method for annealing short complementary oligos to a ssDNA molecule or ssRNA molecule to make the molecule more rigid and increase its persistence length.

FIG. 5 illustrates an exemplary method for capturing single-stranded DNA or single-stranded RNA across a dual nanopore sequencing system, by placing capture tags on the ends of the single-stranded DNA or single-stranded RNA target. Capture tags can include a double-stranded segment followed by a single-stranded tail segment.

FIG. 6 shows an example of capturing one strand of a double-stranded DNA molecule (target strand) or one strand of an RNA/DNA heteroduplex molecule (RNA strand) across a dual nanopore sequencing system, by placing tags on the ends of the molecule, where the strand contains single-stranded tails attached at each end to enable its efficient capture by the two nanopores.

FIG. 7A-C illustrate exemplary methods of library preparation of double-stranded (dsDNA) to install poly-A tails. FIG. 7A shows use of blunt end ligation. FIG. 7B shows use of site-directed sticky end ligation. FIG. 7C shows use of chemical ligation.

FIG. 8A-B illustrate exemplary methods of making ssRNA more rigid with a longer persistence length. FIG. 8A shows use of short random complementary sequences.

FIG. 8B shows use of reverse transcriptase.

FIG. 9A-B illustrate exemplary methods to install polymer tails on single-stranded RNA. FIG. 9A shows enzymatic attachment. FIG. 9B shows attachment of reactive groups followed by chemical ligation of polymer tails.

FIG. 10 shows a dual-biological-nanopore-based protein sequencing system depicting capturing the poly-A₆₀ tails of a peptide-DNA conjugate molecule via both nanopores (readers).

FIG. 11A-E illustrate exemplary capture tags that allow a protein target to be electrophoretically captured by nanopore readers. FIG. 11A shows an exemplary dog bone shaped (>==<) DNA double-stranded elongation capture tag. FIG. 11B shows an exemplary single α-helix (SAH) elongation capture tag. FIG. 11C shows exemplary capture tags attached to the ends of a protein of the same type and length. FIG. 11D shows capture tags attached to the ends of a protein that are of the same type but a different length. FIG. 11E shows exemplary capture tags attached to the ends of a protein that are different types and different lengths.

FIG. 12 shows an exemplary method for attaching adapter DNA (capture tags) onto genomic DNA using blunt-end ligation.

FIG. 13 shows an exemplary method for modifying RNA with homopolymer oligonucleotide tails to improve capture and threading into nanopore readers, with and without the use of reverse transcription.

### DETAILED DESCRIPTION

In alternative embodiments, provided are methods, systems for practicing methods as provided herein, and products of manufacture and kits having contained therein, or comprise, a system as provided herein, wherein the system can comprise: a dual nanopore device; and a tagged target polymer comprising a capture tag at a first distal end and a capture tag at a second distal end, wherein the capture tag comprises a single-stranded tail.

Long polymers, for example, nucleic acids of a few hundred bases or longer in length and peptides and proteins of at least 10 amino acids in length) exhibit an extremely low capture rate by various biological nanopores, under standard experimental conditions, likely due to the extensive secondary structure and inaccessible ends of the DNA. Additionally, the persistence length (the stiffness of a polymer, that describes its behavior as a rigid rod) of the molecule, may not be long enough to bridge across a dual nanopore setup. Methods and compositions described herein enable the persistence length of a polymer to be increased and make the ends of the polymer accessible for capture by nanopore readers.

The technology relates in part to methods and compositions that provide a means of placing tags onto both ends of a polymer molecule to enable the co-capture, threading, and translocation of the tagged polymer molecule across and through the nanopore readers of a dual-nanopore sequencing system and the sequencing of the tagged polymer molecule.

In certain embodiments, once a target polymer is tagged, one end of the tagged polymer can be electrophoretically and/or electroosmotically captured by one nanopore, that bias will be reduced and the other end of the tagged polymer can be captured by the second nanopore. The tags on the polymer directly enable the capture of that end by the nanopores. The tags can also aid in bridging the distance between the two nanopores, such that one tagged end can be captured by one nanopore and the other tagged end can be capture by the other nanopore. Once co-captured, the tagged polymer captured across both nanopores, can be electrophoretically and/or electroosmotically pulled into both nanopores, in order to unfold the target polymer and break up its secondary or tertiary structure, in order to stretch, elongate, linearize, and/or hold taut the target polymer across the two nanopores. Once linearized the tagged polymer can be directionally and controllably driven through one of the nanopores, across the two nanopores, such that it can be directly sequenced. The linearized tagged polymer can be driven back and forth across the two readers in order to re-read the polymer and improve the accuracy of the sequencing reads.

### Target Polymers

In certain embodiments, target polymers can comprise single-stranded DNA, single-stranded RNA, double-stranded DNA, an RNA/DNA heteroduplex, proteins, peptides or other polymers including lipids or carbohydrates etc. In some embodiments, a target polymer comprises a nucleic acid of at least a few hundred nucleotides. A target nucleic acid can be of an actual, average, minimum or maximum length defined by a particular preparation process, including without limitation a physical cleavage process (for example, sonication or other shearing process for a particular period of time) or enzymatic cleavage process (for example, contact with one or more particular endonuclease enzymes for a particular period of time). In certain embodiments, a nucleic acid is of an actual, average, minimum or maximum length of about 100 to about 250 million nucleotides, or is about 100 to about 100,000 nucleotides. In some embodiments, a nucleic acid is of an actual, average, minimum or maximum length of about 100 nucleotides, about 300 nucleotides, about 500 nucleotides, about 1000 nucleotides, about 3000 nucleotides, about 5000 nucleotides, about 10,000 nucleotides, about 30,000 nucleotides, about 50,000 nucleotides, about 100,000 nucleotides, about 200,000 nucleotides, about 300,000 nucleotides, about 400,000 nucleotides, about 500,000 nucleotides, about 600,000 nucleotides, about 700,000 nucleotides, about 800,000 nucleotides, about 900,000 nucleotides, about 1 million nucleotides, about 5 million nucleotides, about 10 million nucleotides, about 25 million nucleotides, about 50 million nucleotides or about 100 million nucleotides. In some embodiments, a target polymer comprises a protein or a peptide of ten or more amino acids. In certain embodiments, a protein or peptide is about 20 to about 40,000 amino acids. A target protein or peptide can be of an actual, average, minimum or maximum length defined by a particular preparation process, including without limitation an enzymatic cleavage process (for example, contact with one or more particular protease enzymes for a particular period of time). In some embodiments, a protein or peptide is of an actual, average, minimum or maximum length of about 10 amino acids to about 40,000 amino acids or about 10 amino acids to about 5,000 amino acids, or about 20 amino acids, about 50 amino acids, about 100 amino acids, about 300 amino acids, about 500 amino acids, about 1,000 amino acids, about 3,000 amino acids, about 5,000 amino acids, about 10,000 amino acids, or about 40,000 amino acids.

A target strand is a strand of a target polymer containing one or more monomers which are to be detected. In certain embodiments, detected monomers are identified. In certain embodiments, a plurality of monomers are detected and identified and a sequence of a target polymer or a partial sequence of a target polymer comprising the identified monomers is determined. In certain embodiments, a target polymer itself is a target strand. For example, for single-stranded RNA, single-stranded DNA, a protein or a peptide, the molecule is a single strand and thus the molecule is a target strand. In certain embodiments, one strand of a double-stranded nucleic acid is a target strand. For example, the sense strand of double-stranded DNA (strand typically presented in the 5′ to 3′ orientation) is a target strand and the RNA strand of an RNA/DNA heteroduplex is a target strand. The complementary strand of a double-stranded DNA molecule or the DNA strand of an RNA/DNA heteroduplex molecule is a non-target strand.

### Capture Tags for Nucleic Acid Target Polymers

### Single-Stranded Tails

In some embodiments, a capture tag comprises a single-stranded tail that promotes efficient capture of a target polymer by a nanopore reader and/or threading through a nanopore reader. In certain embodiments, a single-stranded tail can be of any identity that remains free of secondary structure (including homopolymer A, C, or T; any single-stranded nucleic acid sequence that remains free of secondary structure; abasic sites; modified nucleobases; charged peptide monomers; charged polymers, etc.). In certain embodiments, a single-stranded tail comprises nucleic acid. In some embodiments, the nucleic acid of a single-stranded tail comprises DNA. In some embodiments, the nucleic acid of a single-stranded tail comprises RNA. In some embodiments, a single-stranded tail comprises an unstructured DNA heteropolymer or an unstructured DNA homopolymer. Unstructured refers to a sequence designed such that it does not adopt stable secondary structures such as hairpins, G-quadruplexes, H-DNA, or i-motifs. In some embodiments, a single-stranded tail comprises a homopolymer of A, C or T, abasic sites or modified nucleobases. In some embodiments, a single-stranded tail comprises a non-self complementary, non-homopolymer sequence of more than one type of base, comprising combinations of A, C, T, G, or abasic sites which does not fold up onto itself. In some embodiments, a single-stranded tail comprises a poly A homopolymer.

In certain embodiments, a single-stranded tail comprises a charged polymer. In some embodiments, a single-stranded tail comprises a charged peptide polymer. In some embodiments, a charged peptide polymer comprises polyarginine, polylysine or polyglutamate. In some embodiments, a single-stranded tail comprises a synthetic polymer. In certain embodiments, a synthetic polymer comprises polystyrene sulfonate, polyamine, polyacrylate or polyvinyl sulfonate.

A single-stranded tail regardless of its composition can be of any length that facilitates efficient capture of a target strand by a nanopore reader and/or threading through a nanopore reader. In some embodiments, a single-stranded tail comprises 20 or more nucleotides, amino acids or synthetic monomer units. In some embodiments, a single-stranded tail comprises about 20 to about 1000 nucleotides, amino acids, or synthetic monomer units. In some embodiments, a single-stranded tail comprises about 20 to about 100 nucleotides, amino acids, or synthetic monomer units. In some embodiments, a single-stranded tail comprises 20 or more nucleotides. In some embodiments, a single-stranded tail comprises about 20 to about 1000 nucleotides, about 20 to about 100 nucleotides, about 40 to about 100 nucleotides, about 30 to about 50 nucleotides, or about 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 400, 500, 600, 700, 800, 900, 1000 or more nucleotides.

In some embodiments, a capture tag comprises a single-stranded tail that is attached to a target polymer. In some embodiments, a capture tag comprises a single-stranded tail is synthesized directly from the end of a strand of a target polymer.

Single-stranded tails may be synthesized from the end of the polymer using enzymatic or synthetic means, or they may be synthesized using enzymatic or synthetic (for example, solid-phase DNA synthesis) means and ligated to the target polymer.

Charged polymers are synthesized by radical polymerization of charged monomers. Charged polymers may be comprised of a single monomer or a mixture of two or more monomers.

DNA Duplexes with Single-Stranded Tails

In some embodiments, a capture tag comprises a double-stranded DNA segment with a single-stranded tail, as described above, at one end for efficient nanopore capture and/threading. In some embodiments, the double-stranded DNA segment comprises a chemical functionality for covalent attachment to a target polymer at the other end. In certain embodiments, a double-stranded segment of a capture tag is attached to a DNA or RNA target polymer by chemical or enzymatic ligation.

A double-stranded (DNA duplex) segment can be any length and/or sequence. In some embodiments, a double-stranded DNA segment comprises an actual, average, minimum or maximum length of about 10 to about 10,000 nucleotides, or about 10, 11, 12, 13, 14, 15, 20, 25, 30, 35, 40, 45, 50, 55, 60, 65, 70, 75, 80, 85, 90, 85, 100, 110, 120, 130, 140, 150, 160, 170, 180, 190, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1500, 2000, 2500, 3000, 3500, 4000, 4500, 5000, 5500, 6000, 6500, 7000, 7500, 8000, 8500, 9000, 9500, 10000 nucleotides. In certain embodiments, a double-stranded DNA segment comprises an average, minimum or maximum length of about 10 to about 100 nucleotides, about 10 to about 30 nucleotides, about 15 to about 20 nucleotides or about 10, 11, 12, 13, 14, 15, 16, 17, 18, 19, 20, 21, 22, 23, 24, 25, 26, 27, 28, 29, 30, 40, 50, 60, 70, 80, 90 or 100 nucleotides.

The length of a double-stranded segment of a capture tag is determined in part by the length of the target polymer. In some embodiments, a long target polymer (for example, about 1000 nucleotides) may require a capture tag with a shorter double-stranded segment (for example, about 10 to about 30 nucleotides) as the target polymer itself is of sufficient length to span the gap between the two nanopore readers and the double-stranded segment need not provide elongation, only stability and rigidity. In some embodiments, a short target polymer (for example, about 100 nucleotides) may require a capture tag with a longer double-stranded segment (for example, about 500 to about 1000, or more nucleotides) as the target polymer may be of insufficient length to span the gap between the two nanopore readers and the double-stranded segment provides elongation along with stability and rigidity.

In certain embodiments, the double-stranded segment may be synthesized by preparing two complementary ssDNA strands in lengths of about 10 to about 200 nucleotides (for example, an actual, average, minimum or maximum length of about 10, 20, 30, 40, 50, 60, 70, 80, 90, 100, 125, 150 or 200, or more nucleotides) using solid-phase DNA synthesis, and then annealing them together to form dsDNA. In some embodiments, double-stranded DNA segments may be synthesized and amplified from template double-stranded DNA using the polymerase chain reaction (PCR). In certain embodiments, the single-stranded DNA tail is synthesized as part of the same polymer strand as the double-stranded DNA and annealed to a shorter complementary DNA strand which does not form a duplex with the single-stranded tail. In other embodiments, the single-stranded tail may be synthesized enzymatically from the double-stranded DNA. In some embodiments, the single-stranded tail may be synthesized separately (by for example solid phase DNA synthesis) and ligated to reactive functional groups on the end of one strand in the double-stranded DNA.

In some embodiments, a double-stranded DNA segment of a capture tag comprises a first end, a second end, a capture strand and a complementary strand, at the first end of the double-stranded DNA segment a single-stranded tail extends from the capture strand of the double-stranded DNA segment and a non-complementary overhang or a single-stranded tail extends from the complementary strand or there is no extension from the complementary strand of the double-stranded DNA segment and at the second end of the double-stranded DNA segment the capture strand is configured for attachment to a target strand of a target polymer and the complementary strand is not configured for attachment to a non-target strand if it is present in a target polymer. In certain embodiments, at the second end of the double-stranded DNA segment the capture strand is configured for attachment to a target strand of a target polymer and the complementary strand is configured for attachment to a non-target strand of a target polymer.

In certain embodiments, at the first end of the double-stranded DNA segment the complementary stand is configured to prevent entry of the complementary strand into a nanopore reader. In some embodiments, the complementary strand of the double-stranded DNA segment terminates in a very short non-complementary overhang that prevents its entry into a nanopore reader. In some embodiments, a single-stranded tail that extends from the complementary strand of the double-stranded DNA segment can be very short to prevent its entry into a nanopore reader. In some embodiments, the single-stranded tail that extends from the complementary stand of the double-stranded DNA segment is significantly shorter than the single-stranded tail that extends from the stand of the double-stranded DNA segment and which assists in capture by a nanopore reader. In certain embodiments, the length of an overhang or a single-stranded tail comprising a nucleic acid which extends from the complementary strand can be less than about 20 nucleotides, or can be less than about 15 nucleotides, or can be less than about 10 nucleotides or about 9, 8, 7, 6, 5, 4, 3, 2, 1 nucleotide(s).

In some embodiments, there is no extension from the end of the complementary strand of the double-stranded DNA segment, i.e., no overhang or no single-stranded tail. In some embodiments, a non-complementary overhang or a single-stranded tail that extends from the complementary strand of a double-stranded DNA segment can be attached to a blocking molecule. In certain embodiments, the blocking molecule comprises a biotin/streptavidin complex, an antigen/antibody complex, a nanoparticle, a bulky DNA or RNA structure, a G-quadruplex, an i-motif, a cruciform-forming sequence, a pseudoknot a triple helix, a dendrimer, a polysaccharide, polyethylene glycol, a gold nanoparticle or a polystyrene nanoparticle. A blocking molecule can further ensure that a duplex, a non-complementary overhang or the incorrect single-stranded tail (single-stranded tail attached to a complementary strand) does not enter a nanopore.

Capture tags comprising a double-stranded DNA segment, individually or in combination (when attached to each end of a target polymer) can have a persistence length long enough to span the distance across two adjacent nanopore readers. The free hanging single-stranded tail can help with efficient capturing the target strand of the target polymer by the nanopore reader, while the duplex stabilizes the strand by making it more rigid, and as a result it will have a greater persistence length, thus exposing the free tails from the folded or balled up target polymer to enhance nanopore entry.

Once the leader single-stranded tail portion of a capture tag is captured, the duplex portion of the capture tag can then be mechanically unzipped (because the duplex will not fit through the nanopore) by applying higher potentials, thus, allowing the captured strand to thread through a nanopore. Furthermore, a DNA duplex having a short, noncomplementary overhang or a short single-stranded tail extending from a complementary strand can force unzipping of the DNA to occur outside the pore and ensure that the duplex ends do not enter and obstruct or block the pore reader thus preventing sequencing of the target strand of the target polymer. Also, a short noncomplementary overhang or single-stranded tail extending from a complementary strand with an attached large blocking molecule can ensure the capture tag duplex, or the incorrect strand of the DNA duplex does not enter the nanopore.

The capture tags, single-stranded tail segments, double-stranded DNA segments, extensions, and attached molecules, etc. described herein can be utilized with any of the nucleic acid targets (for example, single-stranded RNA, single-stranded DNA, double-stranded DNA or an RNA/DNA heteroduplex) discussed below.

### Protein or Peptide Capture Tags

In certain embodiments, capture tags comprising single-stranded tails, as described herein, can be attached to protein or peptide targets. In some embodiments, attachment of capture tags to proteins and peptides can be by chemical ligation, enzymatic ligation or combined chemical tagging with subsequent enzymatic ligation.

In some embodiments, a capture tag comprises a double-stranded DNA segment comprising a first end, a second end, a capture strand, and a complementary strand. At the first end of the double-stranded DNA segment a single-stranded tail extends from the capture strand of the double-stranded DNA segment and a non-complementary overhang or a single-stranded tail extends from the complementary strand of the double-stranded DNA segment or there is no extension from the complementary strand of the double-stranded DNA segment. At the second end of the double-stranded DNA segment a single-stranded tail extends from the capture strand of the double-stranded DNA segment or there is no extension from the capture strand of the double-stranded DNA segment. The capture strand is configured to attach to an N terminus or a C terminus of a protein or a peptide. A non-complementary overhang or a single-stranded tail extends from the complementary strand of the double-stranded DNA segment or there is no extension from the complementary strand of the double-stranded DNA segment.

In certain embodiments, at the first end and the second end of the double-stranded DNA segment of a capture tag a complementary stand is configured to prevent entry of the complementary strand into a nanopore reader. In some embodiments, the complementary strand of the double-stranded DNA segment terminates in a very short non-complementary overhang that prevents its entry into a nanopore reader. In some embodiments, a single-stranded tail that extends from the complementary strand of the double-stranded DNA segment is very short which prevents its entry into a nanopore reader. In some embodiments, the single-stranded tail that extends from the complementary stand of the double-stranded DNA segment is significantly shorter than the single-stranded tail that extends from the strand of the double-stranded DNA segment and which assists in capture by a nanopore reader. In certain embodiments, the length of an overhang or a single-stranded tail that comprises nucleic acid and which extends from the complementary strand can be less than about 20 nucleotides, or can be less than about 15 nucleotides, or can be less than about 10 nucleotides or about 9, 8, 7, 6, 5, 4, 3, 2, 1 nucleotide(s). In some embodiments, there is no extension from the end of the complementary strand of the double-stranded DNA segment, i.e., no overhang or no single-stranded tail. In some embodiments, a non-complementary overhang or a single-stranded tail that extends from the complementary strand of the double-stranded DNA segment can be attached to a blocking molecule. In certain embodiments, the blocking molecule comprises a biotin/streptavidin complex, an antigen/antibody complex, a nanoparticle, a bulky DNA or RNA structure, a G-quadruplex, an i-motif, a cruciform-forming sequence, a pseudoknot a triple helix, a dendrimer, a polysaccharide, polyethylene glycol, a gold nanoparticle or a polystyrene nanoparticle. A blocking molecule can further ensure that a duplex, the non-complementary overhang or the incorrect single-stranded tail (single-stranded tail attached to a complementary strand) does not enter a nanopore.

In some embodiments, a double-stranded DNA segment of a capture tag comprises about 1,000 to about 10,000 base pairs, about 1,500 to about 5,000 base pairs or about 1,000, 1,100, 1,200, 1,300, 1,400, 1,500, 1,600, 1,700, 1,800, 1,900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4, 100, 4,200, 4,300, 4,400, 4,500, 4,600, 4,700, 4,800, 4,900, 5,000, 5,500, 6,000, 6,500, 7,000, 7,500, 8,000, 8,500, 9,000, 9,500, 10,000, or more base pairs.

In some embodiments, a capture tag comprises a rigid polymer comprising a first end and a second end. A single-stranded tail extends from the first end of the rigid polymer. A single-stranded tail extends from the second end of the rigid polymer or there is no extension from the second end of the rigid polymer. The second end of the rigid polymer is configured to attach to an N terminus or a C terminus of a protein or a peptide. In certain embodiments, a rigid polymer comprises a polypeptide. In some embodiments, a polypeptide is a single alpha-helix (SAH), a collagen-like helix or a coil-coil structure. In certain embodiments, a polypeptide is a single alpha-helix (SAH) comprising about 3000 amino acids. In certain embodiments, a single alpha-helix (SAH) comprises about 50 to about 3000 amino acids or about 50, 60, 70, 80, 90, 100, 200, 300, 400, 500, 600, 700, 800, 900, 1000, 1200, 1500, 1800, 2000, 2500, or 3000, or more amino acids.

### Capture Tag Length, Composition, and Type

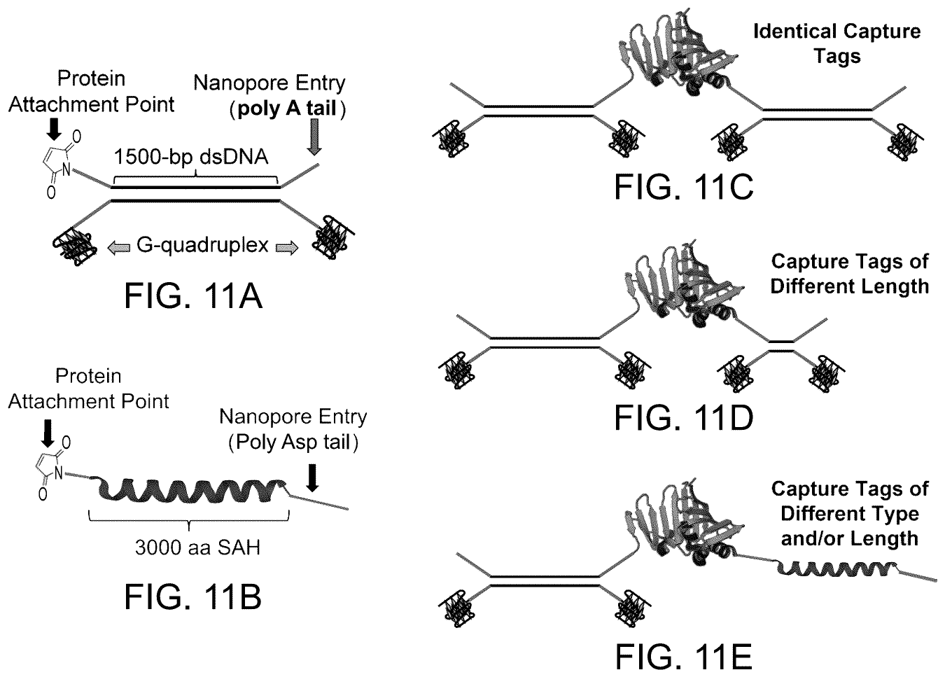

In some embodiments, capture tags attached at the opposite ends of a target polymer molecule are symmetric in length, composition, and type. For example, FIG. 11C shows identical capture tags attached to the C terminus and the N terminus of a protein target.

In some embodiments, capture tags attached at the opposite ends of a target polymer molecule do not have to be symmetric in length, composition, or type.

In certain embodiments, capture tags of the same type but of different lengths can be attached at each end of a target polymer molecule. For example, FIG. 11D shows capture tags of the same type but of different lengths attached to the C terminus and the N terminus of a protein target. For example, it may be appropriate to tag one end of a target protein with a capture tag that contains a single-stranded tail and a very long double-stranded DNA segment to bridge the gap between the two nanopore readers within a dual nanopore reader sequencing system. Because the gap between the two nanopore readers is bridged by a capture tag attached at one end of a protein, it would then only be necessary to have a capture tag attached to the other end of the protein, that contains only a single-stranded tail or a single-stranded tail and only a very short double-stranded segment.

In certain embodiments, a capture tag of the same type but of a different composition can be attached at each end of a target polymer molecule. In some embodiments, a different composition of a capture tag could be a difference in the sequence of a double-stranded segment, a difference in the extension from a complementary strand (a non-complementary overhang, a single-stranded tail or no extension), a difference in the make-up of a single-stranded tail (for example, nucleic acid or synthetic polymer, heteropolymer or homopolymer, etc.) or a difference in the type of blocking molecule attached to a complementary strand.

In certain embodiments, one type of capture tag is attached to one end of a target polymer molecule and a different type of capture tag is attached to the other end of the target polymer molecule. In some embodiments, the capture tags are of the same length.

In certain embodiments, one type of capture tag of with a first length is attached to one end of a target polymer molecule and a different type of capture tag with a second length different from the first length is attached to the other end of the target polymer molecule. For example, FIG. 11E shows a capture tag comprising a double-stranded DNA segment of one length is attached to one end of a protein target and a capture tag comprising a rigid polymer (single α-helix) of a different length is attached to the other end of the protein target.

### Long Single-Strand DNA Capture

In some embodiments, a target polymer is a single-stranded DNA molecule and the capture frequency of the ssDNA is improved by having capture tags comprising single-stranded tails attached to the ends of the single-stranded DNA target. In certain embodiments, the target polymer is a long single-stranded DNA molecule. A long single-stranded DNA molecule typically comprises more than about 100 nucleotides.

In certain embodiments, single-stranded tails are directly attached to the ends of a single-stranded DNA target. In some embodiments, the 5′ and 3′ ends of a single-stranded DNA target can be functionalized with a reactive group. These reactive end groups can be reacted with single-stranded tails that have a reactive partner that specifically reacts with the reactive group at the 5′ or 3′ end of the single-stranded DNA target. In certain embodiments, the target DNA is modified with an orthogonal reactive group (for example, phosphorothioate, amine, azide, or alkyne) and then reacted with a single-stranded tail modified on an end with the orthogonal reaction partner (for example, iodoacetamide, dinitroflorobenzene, azide, or alkyne) (Abe, H.; Kimura, Y., Chemical Ligation Reactions of Oligonucleotides for Biological and Medicinal Applications. Chem Pharm Bull (Tokyo) 2018, 66 (2), 117-122). In some embodiments, a single-stranded tail comprises a single-stranded poly-A tail modified at one end with a reactive partner. In some embodiments, each unique orthogonal reactive group on the 5′ end and the 3′ end of the single-stranded DNA target is chemically ligated to its respective reaction partner on the single-stranded tail that specifically reacts with the unique orthogonal reactive group.

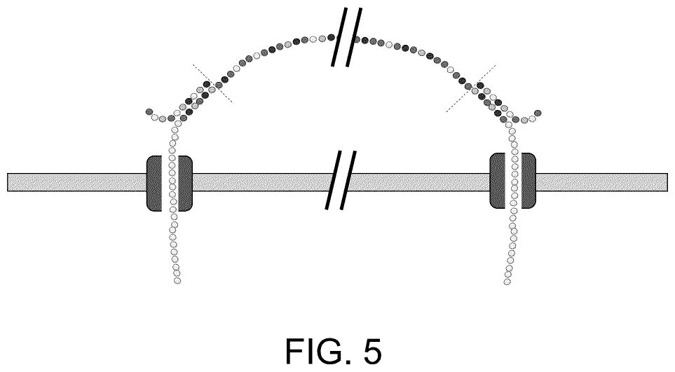

In certain embodiments, the capture frequency of a single-stranded DNA target is improved by modifying the 5′ and/or 3′ end of the strand by adding a capture tag (adaptor) comprising a DNA duplex segment with a single-stranded tail to facilitate capture and entry into the nanopores (see FIG. 5). The duplex can be attached via a two-step ligation conducted in one of two approaches. One approach is to 3′ poly-dA tail the DNA with terminal transferase followed by ligation of a designer duplex DNA with a protruding complementary 3′ poly-dT to functionalize the 3′ end. Next, phosphorylation of the 5′ end typically occurs via polynucleotide kinase, followed by ligation of the second designer duplex to this end. Another approach is direct ligation to the 3′ end with a designer duplex with an available 5′ phosphate group for this reaction. Next, the 5′ end of the target single-stranded DNA typically is phosphorylated with polynucleotide kinase followed by ligation of the designer duplex DNA. For these strategies, the ends of the designer duplex DNAs not involved in the ligation typically are chemically blocked (for example, 3′-ends functionalized as 3′-amine or 2′,3′-dideoxynucleotide and 5′-ends left as hydroxyl groups during solid-phase synthesis) to prevent concatenation of the target nucleic acid.

The adaptors are attached to ssDNA as part of the process to convert dsDNA to ssDNA that will be conducted by fragmentase digestion of dsDNA with adaptors of known sequence formed by solid-phase synthesis. These sequences will either directly install or enable installation via a secondary ligation of a synthetic DNA with a sequence of our choosing that places a biotin on one strand and the 18-mer with a poly-A tail on the other strand. Affinity purification will be used to remove the biotinylated strand while releasing the complementary ssDNA for sequencing that has the known sequences on the ends.

The double-stranded DNA segment can provide stability, rigidity, and elongation, while the single-stranded tail enables the efficient capture of the single-stranded DNA target by both nanopore readers.

In some embodiments, as shown in FIG. 5, a double-stranded segment of a capture tag has a short overhang on a complementary strand which aids in its removal/unzipping from the captured strand as it is electrophoretically and/or electroosmotically driven through either of the nanopores. In some embodiments, the short overhang can be attached to a blocking molecule to prevent the complementary strand from entering a nanopore.

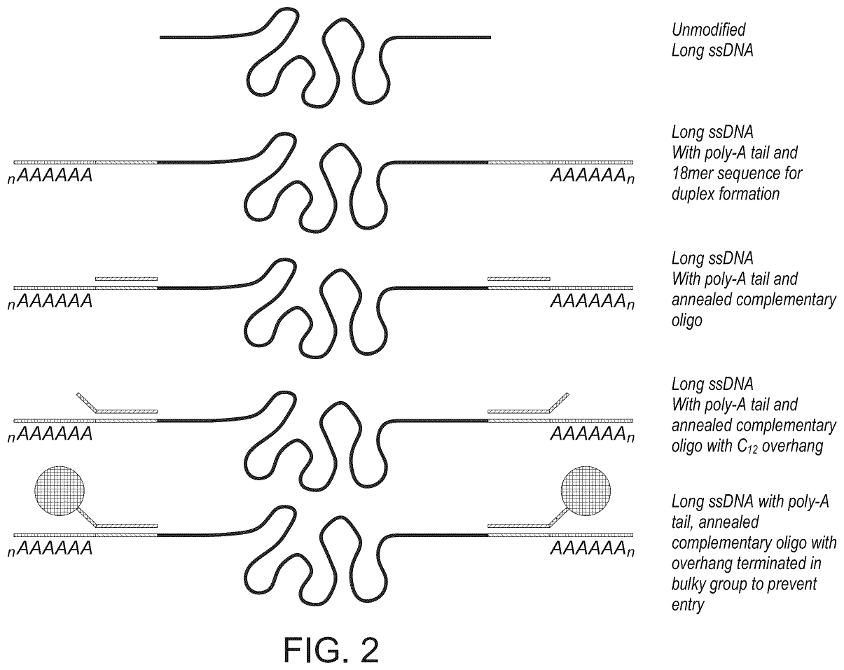

In some embodiments, tagging of single-stranded DNA target can carried out as shown in FIG. 2. For example, an 18-mer sequence for duplex formation attached to a poly-deoxyadenosine (poly-A) tail is attached to the 5′ and the 3′ ends of a single-stranded DNA target. Each poly-A tail independently can be any suitable length, and “n” in FIG. 2 can be an integer between about 1 and 100, and each poly-A tail independently can be about 20 or more nucleotides, about 20 to about 1000 nucleotides, about 20 to about 100 nucleotides, about 40 to about 100 nucleotides, about 30 to about 50 nucleotides, or about 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 400, 500, 600, 700, 800, 900, 1000 or more nucleotides. In certain embodiments, one or more deoxyadenosine monomers in the poly-A tail independently can be replaced by another monomer unit (for example, a deoxycytidine, thymidine, deoxyguanosine, or a synthetic monomer unit). Attachment of the 18-mer sometimes is based on knowing the sequence (i.e., region of interest) to which the poly-dA sequences are attached so that complementary hybridization can be utilized, or the poly-dA segments sometimes are installed via ligation with known complementary sequences to which the 18-mers can hybridize. A complementary oligonucleotide with a C12 overhang can be annealed to the 18-mer attached at the 5′ and 3′ ends of the single-stranded DNA target. The overhang can be attached to a bulky group to prevent entry of the complementary oligonucleotide into the nanopore. In some embodiments, the length of the oligomer sequence for duplex formation, the composition of the single-stranded tail, and the length and composition of the overhang can be any of the embodiments of these features described herein.

In certain embodiments other methods can be used for stabilizing and elongating a long single-stranded DNA target such that the molecule itself, provides the persistence length required to span the distance across two adjacent nanopores (for example, readers) for sequencing.

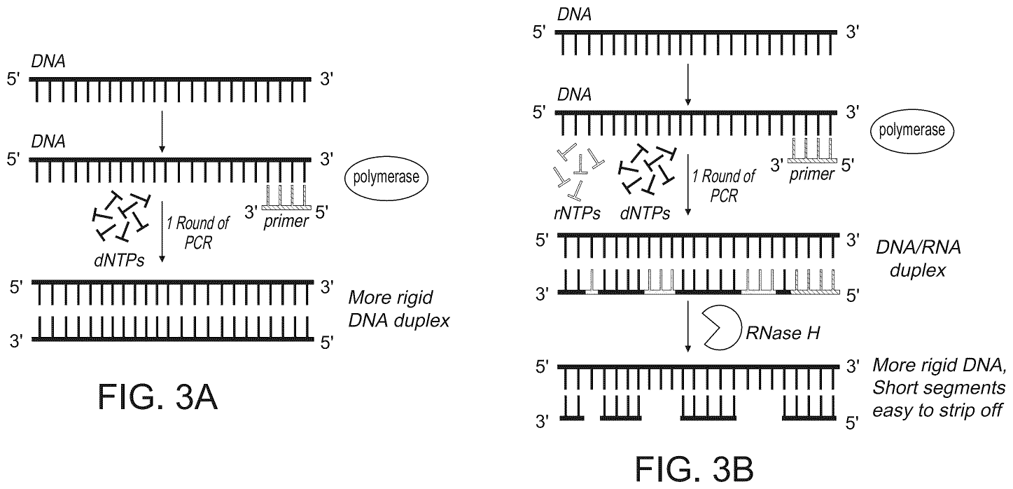

In some embodiments, a long complementary DNA or RNA strand is synthesized and annealed to the template strand. A long continuous complementary DNA strand can be synthesized by performing a single round of PCR on a DNA template strand with 2′-deoxyribonucleotide triphosphates (see FIG. 3A).

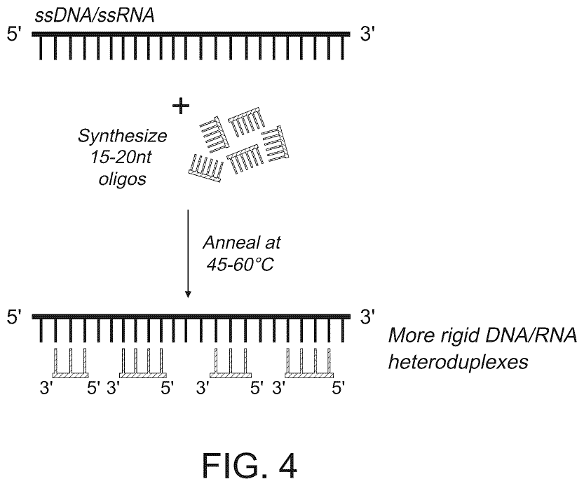

In some embodiments, multiple, complementary short strands are synthesized and annealed onto the template single-stranded DNA target. In certain embodiments, a single round of PCR on a DNA template strand is carried out with addition of a smaller concentration of ribonucleotide triphosphates, thus incorporating ribonucleotides at random locations in the complementary strand. This complementary strand can be selectively nicked to form many short oligos hybridized to the longer target DNA strand by applying RNase H to excise the ribonucleotides (see FIG. 3B). Rnase H is an endoribonuclease that specifically hydrolyzes phosphodiester bonds of RNA that is hybridized to DNA. In some embodiments, a portion of the T nucleotide triphosphates can be substituted with 2′-deoxyuridine nucleotide triphosphates (dU) for random PCR incorporation into the extended strand. The incorporated U nucleotides are substrates for the DNA repair glycosylase UDG and APE1 that are easily cleaved and converted to strand breaks to aid template removal. In some embodiments, if the single-stranded DNA target sequence is known, short oligos (for example, about 15 to about 20 nucleotides) could be designed based on the template and annealed prior to experimental setup to make the single-stranded DNA target more rigid and increase its persistence length, such that it can bridge the gap between two nanopores, in a dual nanopore sequencing system. (see FIG. 4).

As described above, capture tags comprising DNA duplexes could be separated (unzipped) and short oligos be stripped off with higher voltage potentials as they are entering the nanopore reader. The single-stranded tails would remain attached to the ends of the single-stranded DNA target to enable the efficient capture of both ends, by a dual nanopore system.

In some embodiments, to co-capture a tagged single-stranded DNA target across two readers, the tagged single-stranded DNA target, for example having capture tags of double-stranded DNA segments with appended single-stranded tails or having short non-complementary overhangs can be added to a dual nanopore sequencing apparatus (see FIG. 5). In some embodiments, FPGA-controlled dual capture can then be utilized. In some embodiments, the nanopore 1 bias (V₁) can be set to +120 mV or higher (for example, +140 mV, +160 mV, +180 mV, +200 mV, +220 mV, +300 mV) while the nanopore 2 bias (V₂) is set to −10 mV to prevent entry. Once the DNA strand is captured by nanopore 1 (as determined by a decrease in current at nanopore 1 (11) for a preset time), V₁ can immediately be decreased to a potential low enough to hold the DNA strand in place (neither unzipping and translocating or allowing it to eject and escape), while V₂ is set to +120 mV or higher (for example, +140 mV, +160 mV, +180 mV, +200 mV, +220 mV+300 mV) until the free end of the DNA strand via the single-stranded tail is captured by the pore 2. Once dually captured, V₁ and V₂ can be increased to simultaneously pull the captured strand taught and confirm dual capture.

### Long Double-Strand DNA Capture

In certain embodiments, within a dual-nanopore sequencing platform, the tail capture approach described above can be adopted in order to capture long double-stranded DNA, which has a significantly longer persistence length than single-stranded DNA, across two adjacent nanopore readers.

In some embodiments, tagging of a double-stranded DNA target comprises attachment of single-stranded tail directly to the ends of the target strand of the double-stranded DNA target. The 3′-ends of the double-stranded DNA target can be poly-dA tailed with terminal transferase.

In some embodiments, incorporation of capture tags containing the single-stranded tails needed to thread into and through a nanopore can be achieved by using standard library preparation protocols (blunt end ligation, sticky end ligation, TA cloning, or chemical ligation).

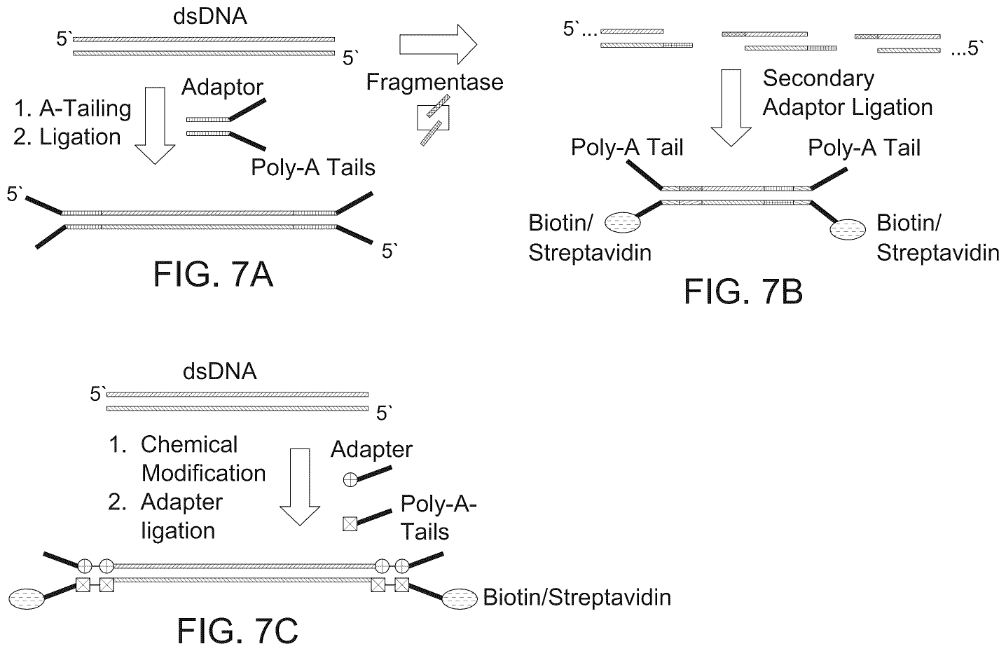

In certain embodiments, tagging of a double-stranded DNA target comprises attachment of a capture tag comprising a double-stranded DNA segment with a single-stranded tail to each end of the double-stranded DNA target. In some embodiments, a Y-shaped capture tag comprised of a double-stranded DNA segment with two single-stranded DNA poly-A tails attached at one end (=<) can be attached at each end a double-stranded DNA target using blunt end ligation (see FIG. 7A). The Y-shaped adapter is prepared by synthesizing two single-stranded DNA strands, which contain tails and mutually complementary regions such that when they are mixed together they hybridize and form a duplex structure with two individual tails extending from the each strand on the same end of the duplex. When one single-stranded DNA tail enters the reader, the other tail forming the capture tag (Y-shaped adaptor) will be blocked from entry, and the duplex will be forced to unzip on the outside of the reader. For blunt-end ligation (as depicted in FIG. 7A), the double-stranded segments of the capture probe attached at each end of the double-stranded DNA target often are the same.

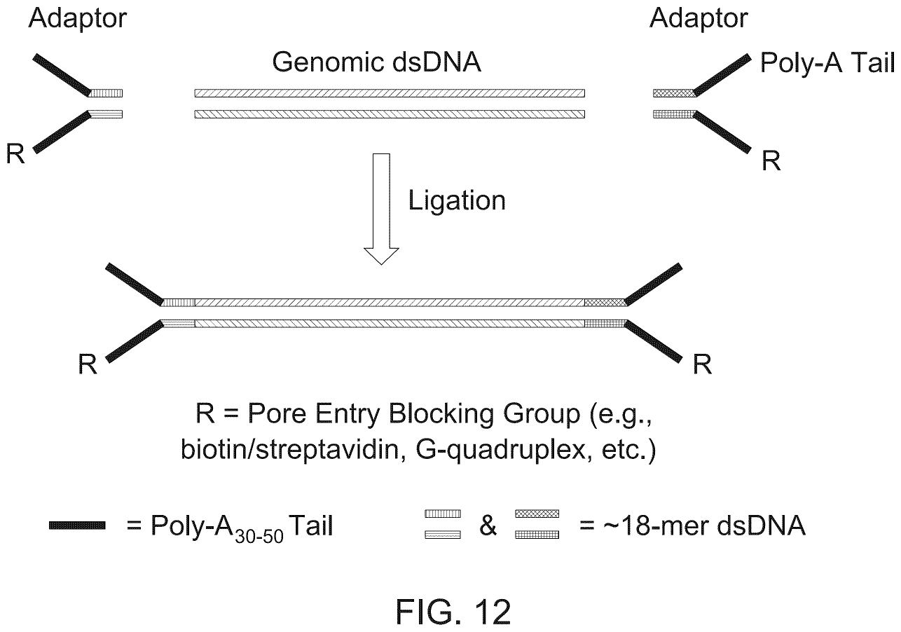

In some embodiments, capture tags (adaptor DNA) can be attached onto genomic DNA (double-strand DNA target) using blunt-end ligation. In certain embodiments, DNA adapters can have an 18 bp duplex region with a blunt end for ligation onto genomic DNA, and flexible homopolymer single-stranded DNA tails (for example, about 30 to about 50 nucleotides) to assist capture, threading, and unzipping of the genomic DNA by the nanopore reader. In certain embodiments, the capture tag attached at each end of the double-stranded DNA target comprise different 18 bp duplex regions (see, for example, FIG. 12). In this case, the ligation is the same, but the tails are distinct in that one tail contains a bulky blocking group to prevent nanopore entry, and the other tail has no blocking group and is designed to enter the nanopore. In certain embodiments, one terminal end of a homopolymer tail may be modified with a bulky blocking group to prevent entry into the nanopore reader and assist unzipping of the genomic duplex DNA. In some embodiments, one terminal end of a homopolymer tail may be made very short, to prevent its entry into the nanopore reader.

In some embodiments, to ensure only one strand of the double-stranded DNA target (i.e., the target strand) has accessible poly-A, and to ensure the dual capture of that target strand, the double-stranded DNA target is subjected to fragmentase digestion with dual custom synthetic 5′ adaptors to function as handles for secondary ligation to install free poly-A tails on the ends of one strand (the target strand) and a biotin/streptavidin blocked overhang on the other strand (non-target strand) (see FIG. 7B). The fragmentase approach is traditionally used for small read sequencing; however, greater than 2000 bp fragments can be generated by decreasing the reaction time and fragmentase concentration. (Syed, F.; Grunenwald, H.; Caruccio, N., Next-generation sequencing library preparation: simultaneous fragmentation and tagging using in vitro transposition. Nat. Methods 2009, 6 (11), i-ii). This approach will facilitate working with real-world samples with unknown sequences. Once the target strand of the double-stranded DNA is captured via both readers, the complementary strand (non-target strand) will be completely unzipped (via voltage induced translocation), leaving the target strand held taught/linearized across the two readers, readying it for sequencing.

In some embodiments, enzymatic ligation can be used to attach capture tags to a double-stranded DNA target. In certain embodiments, the strands of a double-stranded segment of a capture tag and the strands of a double-stranded DNA target can have the correct chemical functional groups for ligase-catalyzed covalent attachment of the strands. In the case of ligase attachment, it is not functional groups, but specific sticky end sequences left on the target genomic DNA that facilitate attachment. For example, double-stranded DNA target can be cleaved with a restriction enzyme, leaving sticky ends with a specific sequence. Single-stranded DNA synthesized via solid phase DNA synthesis can be annealed together to form double-stranded tags. These capture tags have sticky ends that are complementary with the sticky ends of the target DNA produced by the restriction enzymes, which allows the tags to be ligated to the target DNA at the sticky ends using DNA ligase.

In some embodiments, chemical ligation can be used to attach capture tags to a double-stranded DNA target. In certain embodiments, the strands of the double-stranded DNA target are modified with an orthogonal reactive group (for example, phosphorothioate, amine, azide, or alkyne) and then reacted with a adaptor modified on the correct end with the orthogonal reaction partner (for example, iodoacetamide, dinitroflorobenzene, azide, or alkyne) (see FIG. 7C). (Abe, H.; Kimura, Y., Chemical Ligation Reactions of Oligonucleotides for Biological and Medicinal Applications. Chem Pharm Bull (Tokyo) 2018, 66 (2), 117-122). In some embodiments, an adaptor comprises a single-stranded poly-A tail modified at an end to react with a reactive partner at the ends of the target strand of the double-stranded DNA target. In some embodiments, an adaptor comprises a single-stranded tail modified at an end to react with reactive partner on the ends of the complementary strand of the double-stranded DNA target (non-target strand). In some embodiments, a blocking molecule, such as biotin/streptavidin, can be attached to an end of the single-stranded tail attached to the ends of the the non-target strand. In some embodiments, attachment of a single-stranded poly-A tail to the ends of a target strand of a double-stranded DNA target results in a capture tag at each end of the double-stranded target DNA. Formation of the covalent linkage will place the nanopore entry part (single-stranded tail) of the tagged dsDNA opposite of the attachment point (to the double-stranded DNA target) to facilitate the preferred strand entry, orientation and elongation for sequencing.

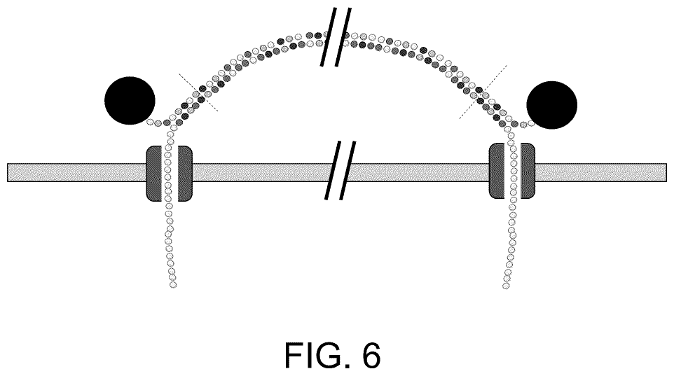

In some embodiments, capturing one strand of a double-stranded DNA molecule across a dual nanopore sequencing system, comprises placing capture tags on the ends of the molecule, where the target single-strand contains single-stranded tails to enable its efficient capture by the two nanopores (see FIG. 6). In some embodiments, capture tags consist of a double-stranded segment followed by a single-stranded tail segment, with the single-stranded tail segment extending out from the target strand of the double-stranded DNA segment. The double-stranded segment of the capture provides stability, rigidity, and elongation, while the single-stranded segments enable the efficient capture of the target strand by both readers. In certain embodiments, the double-stranded segment of the tag has a short overhang that terminates in a large entity, such as a biotin/streptavidin complex, antigen/antibody complex, nanoparticles, bulky DNA or RNA structures such as G-quadruplexes, i-motifs, cruciform-forming sequences, pseudoknots, triple helices, dendrimers, polysaccharides, polyethylene glycol, gold nanoparticles or polystyrene nanoparticles to further ensure the duplex, or the overhang does not enter the nanopore. In some embodiments, either the large entities and/or the non-complementary portion of the double-stranded segment of the tag could be removed.

In some embodiments, to co-capture tagged DNA across two readers, the double-stranded DNA target with appended single-stranded tails will be added to the dual nanopore sequencing apparatus. In some embodiments, FPGA controlled dual capture can then be utilized. In certain embodiments, the nanopore 1 bias (V₁) will be set to +120 mV or higher (for example, +140 mV, +160 mV, +180 mV, +200 mV, +220 mV+300 mV) while the nanopore 2 bias (V₂) is set to −10 mV to prevent entry. Once the DNA strand is captured by the nanopore 1 (as determined by a decrease in current at nanopore 1 (11) for a preset time), V₁ will immediately be decreased to a potential low enough to hold the DNA strand in place (neither unzipping and translocating or allowing it to eject and escape), while V₂ is set to +120 mV or higher (for example, +140 mV, +160 mV, +180 mV, +200 mV, +220 mV+300 mV) until the free end of the target DNA strand via the single-stranded tail is captured by the pore 2. Once dually captured, V₁ and V₂ can be increased to simultaneously pull the captured target strand taught and confirm dual capture.

### Long Strand RNA Capture

In some embodiments, a target polymer is a single-stranded RNA molecule and the capture frequency of the single-stranded RNA is improved by having capture tags comprising single-stranded tails attached to the ends of the single-stranded RNA target. In certain embodiments, the target polymer is a long single-stranded RNA molecule. A long single-stranded RNA molecule typically comprises more than about 100 nucleotides.

In some embodiments, there can be direct attachment of a single-stranded tail to the ends of the target RNA strand. In some embodiments, single-stranded tails are attached using enzymatic attachment. In some embodiments, single-stranded tails are attached using enzymatic attachment of reactive groups on the ends of the RNA followed by chemical ligation of polymer tails.

In some embodiments, tagging native RNA can utilize terminal groups to selectively attach a single-stranded tail to assist entry into the nanopore. In certain embodiments, tagging of the native RNA involves the 3′-poly-A tail and 5′ cap on mRNA. In certain embodiments, one strategy is to oxidize the glycol present on the 5′-N7-methylguanosine cap with periodate to generate a bisaldehyde that can be reacted upon with aldehyde-specific reactions (for example, O-alkylhydroxylamines to yield oxime ethers, alkylamine addition to the aldehyde followed by reduction of the Schiff's base to yield a stable carbon-nitrogen attachment, aldehyde reaction with hydrazine to yield a hydrazone. The aldehyde-specific reagents can be synthesized at the appropriate end of the single-stranded DNA tail. The alkylamine is available as a commercial phosphoramidite for attachment to the 5′-end of the single-stranded DNA. The O-alkylhydroxylamine and hydrazine can be attached to either end of a single-stranded DNA that is terminated with an alkyne via the click reaction. This can be achieved using commercially available bisfunctionalized alkyl groups (i.e., azide and O-hydroxylamine or azide and hydrazine).

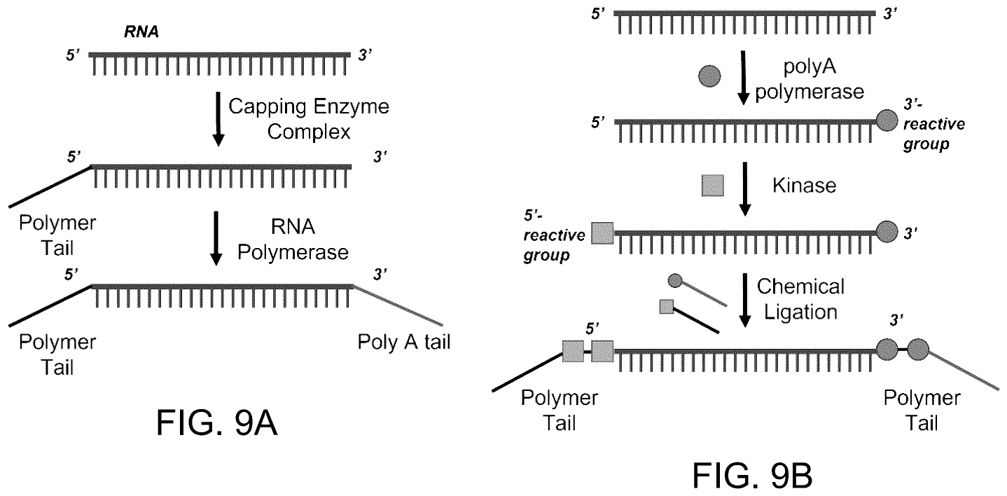

In certain embodiments, RNA 5′ caps (for example, mRNA, snoRNA, or piwiRNA) can be enzymatically removed with a commercially available mRNA decapping enzyme to yield a 5′ phosphate that can be ligated to a single-stranded adaptor. For RNAs that contain a 3′ poly-A tail (for example, mRNA), that 3′ tail can be used as a single-stranded capture tag. For RNAs that do not contain a 3′ poly-A tail (for example, tRNA or rRNA), the homopolymer poly-A tail can be installed enzymatically with a commercially available poly-A polymerase. A non-limiting example in which a RNA 5′ cap is removed is illustrated in FIG. 9A.

In some embodiments, the 5′ and 3′ ends of a single-stranded RNA target can be functionalized with an orthogonal reactive group using enzymatic attachment of reactive groups (for example, poly-A polymerase insertion of a clickable A nucleotide on the 3′ end, and kinase insertion of a clickable phosphate on the 5′ end). These reactive end groups can be reacted with tagging polymer strands (single-stranded tails) that possess reactive partners to attach the polymer tails to a single-stranded RNA target by chemical ligation and to ensure proper orientation of the strands relative to one another for dual nanopore entry (see FIG. 9B). For instance, the 3′ end can be functionalized with a 3′-azido nucleotide that provides a clickable handle to add an alkyne terminated single-stranded DNA. The 5′-end of the target DNA can be functionalized with a phosphorothioate that reacts selectively with synthetic, single-stranded DNA terminated in a maleimide group for selective thiol maleimide covalent attachment.

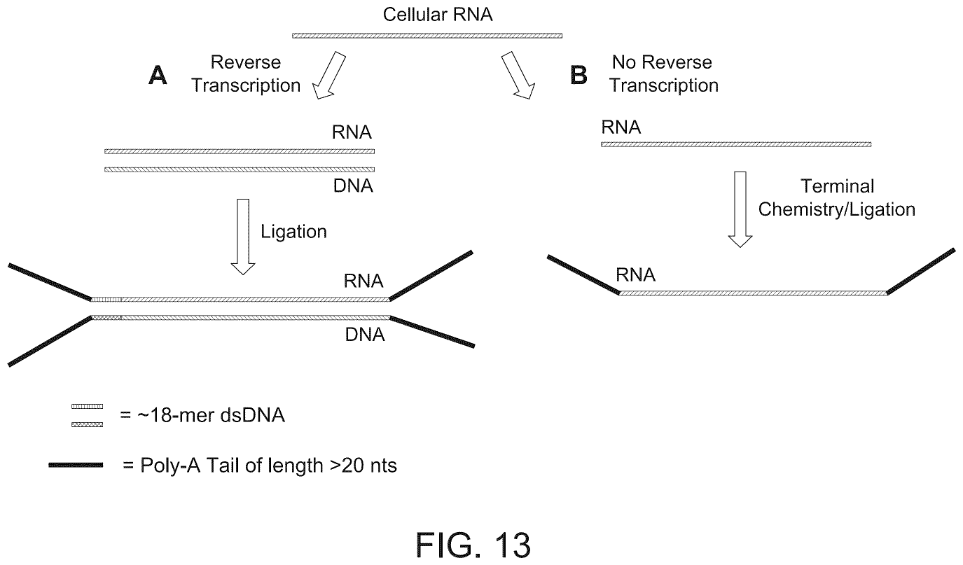

In some embodiments, tagging native RNA is carried out by modification of the ends of the single-stranded RNA with homopolymer tails using chemical modification or ligation. (see FIG. 13B, right panel). For example, the 3′ end can be functionalized with a 3′-azido nucleotide that provides a clickable handle to add an alkyne terminated single-stranded DNA. The 5′-end of the target RNA can be functionalized with a phosphorothioate that reacts selectively with synthetic, single-stranded DNA terminated in a maleimide group for selective thiol maleimide covalent attachment. For capped RNAs, enzymatic removal of the cap followed by dephosphorylation with commercial enzymes can yield an acceptable end for the chemical functionalization described.

In some embodiments, tagging native RNA is carried out by placing tags on the ends of the single-stranded RNA target that comprise a double-stranded segment followed by a single-stranded tail segment (see FIG. 5). For instance, the 3′ end can be functionalized with a 3′-azido nucleotide that provides a clickable handle to add an alkyne-terminated double-stranded DNA with the appropriate single-stranded tails. The 5′-end of the target DNA can be functionalized with a phosphorothioate that reacts selectively with synthetic, double-stranded DNA terminated in a maleimide group with the appropriate single-stranded tails for selective thiol maleimide covalent attachment.

The double-stranded segment of a capture tag provides stability, rigidity, and elongation, while the single-stranded tail segments enable the efficient capture of the RNA target by both readers. In some embodiments, the double-stranded segment of a capture tag has a short overhang extending from a complementary strand of the double-stranded segment which aids in its removal/unzipping from the captured RNA strand as it is electrophoretically and/or electroosmotically driven through either of the nanopores (see FIG. 5). In some embodiments the short overhang can be attached to a blocking molecule to prevent the complementary strand from entering a nanopore.

In some embodiments tagging native RNA is carried out as shown in FIG. 2. The 18-mer can be attached in one of two manners, for example. The first is if the target RNA sequence is known, the 18-mers can be synthesized with known complementarity to the sequences. The second approach includes ligation of the duplexes with the single-stranded tails, as depicted in FIG. 13A, for example. A complementary oligonucleotide with a C12 overhang can be annealed to the 18-mer attached at the 5′ and 3′ ends of the single-stranded RNA target. The overhang can be attached to a bulky group to prevent entry of the complementary oligonucleotide into the nanopore. The length of the oligomer sequence for duplex formation, the composition of the single-stranded tail, and the length and composition of the overhang can be according to an embodiment described herein.

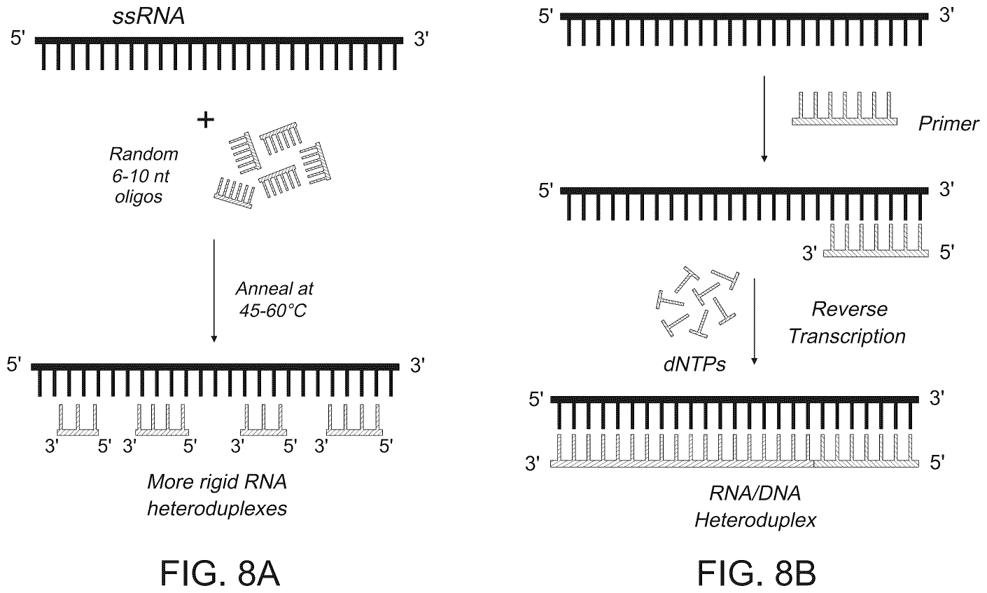

In certain embodiments, to disrupt and linearize single-stranded RNA intramolecular structures and increase single-stranded RNA persistence length, an excess of complementary randomized DNA sequences (about 6 to about 10 nucleotides in length) can be annealed, thus creating a linearized, long-persistence length RNA: DNA heteroduplex (see FIG. 8A). Similarly, a minimal library preparation protocol can be utilized, using reverse transcriptase to generate a complementary DNA strand (cDNA) to yield a RNA: DNA heteroduplex (see FIG. 8B). Alternatively, if the single-stranded RNA sequence is known, short oligos (about 15 to about 20 nucleotides in length) could be designed based on the template and annealed prior to experimental setup to make the single-stranded RNA molecule more rigid and increase its persistence length, such that it can bridge the gap between two nanopores, in a dual nanopore sequencing system. (see FIG. 4). In some embodiments, these heteroduplexes or partial heteroduplexes can be modified with single-stranded tails (for example, single-stranded DNA homopolymer tails) to increase capture efficiency.

In some embodiments, following reverse transcription to synthesize a complementary DNA strand to form an RNA/DNA heteroduplex capture tags comprising short (˜18 bp) double-stranded DNA segments with homopolymer tails (>20 nucleotides) are attached to one end of the RNA-DNA heteroduplex using blunt-end ligation. Homopolymer tails are attached to the opposite end by ligation (onto the DNA strand) and/or synthesis/elongation (onto the RNA strand). (See FIG. 13, left panel).

In some embodiments, after converting a single-stranded RNA to an RNA/DNA heteroduplex molecule. capturing the RNA strand of an RNA/DNA heteroduplex molecule across a dual nanopore sequencing system comprises placing capture tags on the ends of the molecule, so that the target single-strand (RNA strand) contains single-stranded tails to enable its efficient capture by the two nanopores (see FIG. 6). In some embodiments, the capture tags comprise a double-stranded segment followed by a single-stranded tail segment. After attachment of the capture tags the single-stranded tail segments extend out from the target strand (RNA strand) of the RNA/DNA heteroduplex molecule. For example, as illustrated in FIG. 13, dsDNA capture tags can be ligated onto the RNA/DNA heteroduplex using blunt-end ligation.

The double-stranded segment provides stability, rigidity, and elongation, while the single-stranded tail segments enable the efficient capture of the RNA target strand by both readers. In certain embodiments, the double-stranded segment of a capture tag has a short overhang that terminates in a large entity, such as a biotin/streptavidin complex, antigen/antibody complex, nanoparticles, bulky DNA or RNA structures such as G-quadruplexes, i-motifs, cruciform-forming sequences, pseudoknots, triple helices, dendrimers, polysaccharides, polyethylene glycol, gold nanoparticles or polystyrene nanoparticles to further ensure that the duplex, or the overhang does not enter the nanopore. In some embodiments, either the large entities and/or the non-complementary portion of the double stranded segment of the tag could be removed.

In some embodiments, to co-capture tagged single-stranded RNA across two readers, the single-stranded RNA with appended single-stranded tails can be added to a dual nanopore sequencing apparatus. In some embodiments, FPGA-controlled dual capture can then be utilized. In certain embodiments, the nanopore 1 bias (V₁) can be set to +120 mV or higher (for example, +140 mV, +160 mV, +180 mV, +200 mV, +220 mV, +300 mV) while the nanopore 2 bias (V₂) is set to −10 mV to prevent entry. Once the RNA strand is captured by nanopore 1 (as determined by a decrease in current at nanopore 1 (11) for a preset time), V₁ can immediately be decreased to a potential low enough to hold the RNA strand in place (neither unzipping and translocating or allowing it to eject and escape), while V₂ is set to +120 mV or higher (for example, +140 mV, +160 mV, +180 mV, +200 mV, +220 mV+300 mV) until the free end of the RNA strand via the single-stranded tail is captured by the pore 2. Once dually captured, V₁ and V₂ can be increased to simultaneously pull the captured RNA strand taught and confirm dual capture.

### Protein/Peptide Capture

In some embodiments, a target polymer is a protein or peptide and capture of the protein or peptide is enabled by having capture tags comprising single-stranded tails attached to the ends of the protein or peptide target.

Because proteins and peptides do not have a polymer backbone with intrinsic charge like DNA and RNA, their net charge may vary significantly based on their amino acid content. In some embodiments, the C- and N-termini of the protein or peptide may be first ligated to a charged polymer (single-stranded tail) to increase its charge density and facilitate electrophoretic capture and translocation through a nanopore reader. In certain embodiments, the polymer may comprise, but is not limited, to a polypeptide (for example, polyarginine, polylysine, polyglutamate), a DNA polymer, an abasic DNA homopolymer (for example, a tetrahydrofuran spacer that models abasic sites), or a synthetic polymer (for example, polystyrene sulfonate, polyallylamine, polyacrylate, polyvinyl sulfonate).

In some embodiments, the length of a single-stranded tail may comprise about 5 to about 10000 monomer units, about 100 to about 1000 monomer units or about 5, 6, 7, 8, 9, 10, 11, 12, 13, 14, 15, 20, 25, 30, 35, 40, 45, 50, 55, 60, 65, 70, 75, 80, 85, 90, 85, 100, 110, 120, 130, 140, 150, 160, 170, 180, 190, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975,1000, 1500, 2000, 2500, 3000, 3500, 4000, 4500, 5000, 5500, 6000, 6500, 7000, 7500, 8000, 8500, 9000, 9500, 10000 monomer units. The length can be chosen to facilitate high ligation coupling yield and high capture rates by the nanopore readers.

In some embodiments, charged surfactants (for example, sodium dodecyl sulfate) may be added to the fluid containing a protein or peptide target to increase the charge density of the protein or peptide via adsorption to facilitate capture and translocation through the nanopore readers. The added surfactant also serves to denature the protein or peptide secondary structure, facilitating unfolding, stretching, and translocation through the nanopore readers. In certain embodiments, other denaturants, including but not limited, to urea or guanidinium chloride may be added to the fluid to denature the protein or peptide secondary structure to facilitate unfolding, stretching, and translocation through the nanopore readers. In some embodiments, denaturants are utilized in addition to surfactants.

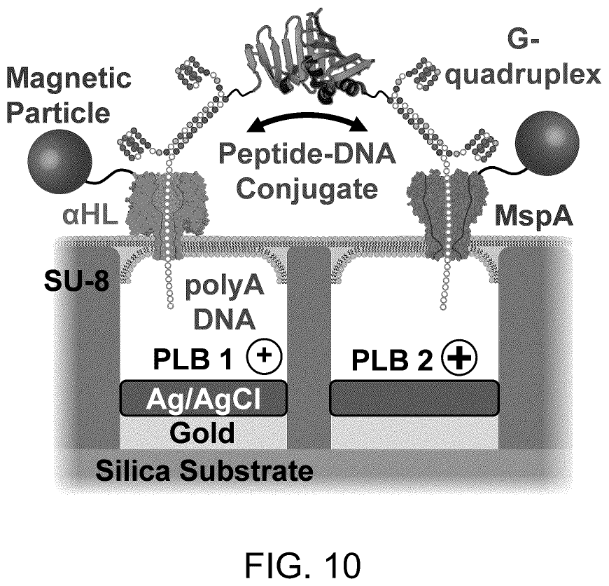

In order to co-capture a protein or peptide target across two nanopore readers, charged capture tags need to be incorporated onto both the N- and C-termini of the protein or peptide target for two reasons: (1) to initially electrophoretically drive the protein or peptide to and capture it in one nanopore reader, and (2) once captured by one nanopore reader, a long enough persistence length is needed to allow the other terminal capture tag to be captured by the other nanopore reader, such that the two capture tags/ends can be pulled against one another, denaturing, elongating and stretching the protein or peptide target into a nearly linear strand, readying it for controlled translocation and sequencing. FIG. 10 illustrates capturing of the single-stranded tails of a protein/peptide-DNA conjugate molecule via both nanopore readers of a dual-biological-nanopore-based protein sequencing system.

In some embodiments, tagging of the N and C termini of a protein or peptide is with capture tags comprising a charged polymer (single-stranded tail). In certain embodiments, the capture tags attached to both the N and C terminus of a protein, or a peptide comprise a rigid, long double-stranded DNA segment, followed by a single-stranded tail segment, which can be electrophoretically captured a nanopore reader. In certain embodiments, capture tags comprise two dog bone shaped (>==<) ds-ssDNA synthetic constructs ligated to the C and N terminal ends of a protein or peptide target. (see FIG. 11A) Use of a capture tag with a long double-stranded DNA segment will allow the protein or peptide target to bridge the reader-to-reader distance to directly enable full length individual reads of the protein or peptide target (end-to-end), as well as enable the ability to multipass/re-read the protein or peptide target if needed. In certain embodiments, a long double-stranded DNA segment of a capture tag comprises about 500 to about 10,000 bp, about 2,500 to about 7,500 bp or about 500, 1000, 1500, 2000, 2500, 3000, 3500, 4000, 4500, 5000, 5500, 6000, 6500, 7000, 7500, 8000, 8500, 9000, 9500, 10000 bp.

A single-stranded tail attached to a capture strand at a first end of a double-stranded segment of a capture tag is for selective entry into nanopore reader (nanopore entry). In certain embodiments, the single-stranded tails can have any of the structures described herein for a single-stranded tail that are appropriate for use with a target protein or peptide. In some embodiments, the single-stranded ends (tails) of the adaptors (capture tags) attached to the capture strand of a double-stranded segment can be comprised of unstructured heteropolymer or homopolymer DNA. Unstructured refers to the sequence of DNA attached designed such that it does not adopt stable secondary structures such as hairpins, G-quadruplexes, H-DNA, or i-motifs. In certain embodiments, a single-stranded tails can be a poly-A tail. In certain embodiments, the single-stranded tails can comprise about 20 to about 1000 nucleotides, about 20 to about 100 nucleotides, about 40 to about 60 nucleotides or about 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 350, 400, 450, 500, 550, 600, 650, 700, 750, 800, 850, 900, 950, 1000 nucleotides.

In some embodiments, a double-stranded segment of a capture tag comprises a first end, a second end, a capture strand (strand that has at one end the single-stranded tail responsible for selective entry into nanopore reader and at the other end attaches to the N terminus or the C terminus of a protein or peptide target) and a complementary strand. At the first end of the double-stranded DNA segment a single-stranded tail extends from the capture strand of the double-stranded DNA segment and a non-complementary overhang or a single-stranded tail extends from the complementary strand of the double-stranded DNA segment or there is no extension from the complementary strand of the double-stranded DNA segment. At the second end of the double-stranded segment a single-stranded tail extends from the capture strand of the double-stranded DNA segment or there is no extension from the capture strand of the double-stranded DNA segment. The capture strand is configured to attach to an N terminus or a C terminus of a protein or a peptide. A non-complementary overhang or a single-stranded tail extends from the complementary strand of the double-stranded DNA segment or there is no extension from the complementary strand of the double-stranded DNA segment.

In some embodiments, at the second end of the double-stranded segment of a capture tag the capture strand (or an extension of the capture strand, for example, overhang or single-stranded tail) can be terminated in a reactive group for attachment to the N or C terminus of a protein or peptide target. In certain embodiments, a reactive group comprises maleimide.

In certain embodiments, single-stranded tails attached to the ends of a complementary strand of a double-stranded DNA segment of a capture tag can have any of the structures described herein for a single-stranded tail that are appropriate for use with a target protein or peptide. In certain embodiments, the single-stranded tails at the ends of the complementary strand of the double-stranded DNA segment can be unstructured. In some embodiments, the single-stranded tails at the ends of the complementary strand of a double-stranded DNA segment can comprise DNA. In certain embodiments, the single-stranded tails at the ends of the complementary strand of the double-stranded DNA segment can comprise about 20 to about 1000 nucleotides, about 20 to about 100 nucleotides, about 40 to about 60 nucleotides or about 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 350, 400, 450, 500, 550, 600, 650, 700, 750, 800, 850, 900, 950, 1000 nucleotides. In certain embodiments, the single-stranded tails attached to the ends of the complementary strand of the double-stranded DNA segment can comprise an unstructured charged polymer of similar length. In some embodiments, at the first and the second end of the double-stranded DNA segment a non-complementary overhang or a single-stranded tail extends from the complementary strand and the non-complementary overhang or the single-stranded tail is attached to a blocking molecule.

that prevents entry of the ends of the complementary strand into a nanopore and can promote mechanical removal of the complementary strand from the capture strand that occurs during translocation of the capture strand through the nanopores. In certain embodiments, a blocking molecule comprises a denaturant-resistant G-quadruplex forming DNA sequence. (Zhang, W.; Liu, M.; Lee, C.; Salena, B. J.; Li, Y., Serendipitous discovery of a guanine-rich DNA molecule with a highly stable structure in urea. Scientific reports 2018, 8 (1), 1-8). In certain embodiments, bulky macromolecules or nanoparticles that block the complementary strand could be used. In some embodiments, the bulky macromolecules or nanoparticles can include a biotin/streptavidin complex, antigen/antibody complex, bulky DNA or RNA structures such as i-motifs, cruciform-forming sequences, pseudoknots, triple helices, dendrimers, polysaccharides, polyethylene glycol, gold nanoparticles or polystyrene nanoparticles.

In some embodiments, DNA tags can be commercially synthesized by a combination of chemical and enzymatic steps to include a 5′-maleimide, NHS-ester, carboxy, peptide enzymatic ligation tag (Tan, D. J. Y.; Cheong, V. V.; Lim, K. W.; Phan, A. T., A modular approach to enzymatic ligation of peptides and proteins with oligonucleotides. Chemical Communications 2021, 57 (45), 5507-5510), or other reactive functional groups for ligation to amino acids of the target protein or peptide. The judicious design of the Y-shaped adaptor arms relative to the capture strand with the maleimide at one end will ensure that only the protein-DNA covalently linked capture strand enters the nanopore readers and will facilitate mechanical unzipping of the blocked (by G-quadruplex or other bulky molecules) complementary strand. In certain embodiments, longer variations and/or alternatives of these dog bone shaped (>==<) adapters can be utilized if necessary.

In certain embodiments, capture tags for protein or petptide target capture by nanopore readers can comprise other rigid polymers with long persistence length (see FIG. 11B). In some embodiments, a rigid polymer comprises a peptide that forms a single α-helices (SAH). In certain embodiments, a SAH capture tag comprises a repeating polypeptide sequence that folds into a rigid α-helical structure, which has a long persistence length comparable to double-stranded DNA (about 20 to about 100 nm) that can span the gap between nanopores. (Choe, S.; Sun, S. X., The elasticity of alpha-helices. The Journal of chemical physics 2005, 122 (24), 244912; Barnes, C. A.; Shen, Y.; Ying, J.; Takagi, Y.; Torchia, D. A.; Sellers, J. R.; Bax, A., Remarkable Rigidity of the Single α-Helical Domain of Myosin-VI As Revealed by NMR Spectroscopy. Journal of the American Chemical Society 2019, 141 (22), 9004-9017). SAH-forming peptides can be captured and translocated through nanopore readers much like single-stranded DNA. (Sutherland, T. C.; Long, Y.-T.; Stefureac, R.-I.; Bediako-Amoa, I.; Kraatz, H.-B.; Lee, J. S., Structure of Peptides Investigated by Nanopore Analysis. Nano Letters 2004, 4 (7), 1273-1277; Stefureac, R.; Long, Y.-t.; Kraatz, H.-B.; Howard, P.; Lee, J. S., Transport of α-Helical Peptides through α-Hemolysin and Aerolysin Pores. Biochemistry 2006, 45 (30), 9172-9179). Such a repeating polypeptide sequence sometimes is about 50 amino acids to about 6,000 amino acids, about 500 to about 5,000 amino acids in length, about 1,000 amino acids to about 4,000 amino acids in length, about 2,000 amino acids to about 4,000 amino acids in length of about 3,000 amino acids in length

In some embodiments, a rigid polymer comprises other structural motifs including collagen-like helices and coil-coil structures.

In certain embodiments, a first terminal end of a rigid polymer comprises a flexible charged polypeptide, polymer, or single-stranded DNA tail to enhance the capture rate by a nanopore reader. In some embodiments, a first terminal end of a rigid polymer comprises an unstructured homopolymer tail for entry into a nanopore. In some embodiments, an unstructured homopolymer tail comprises poly-Asp. In some embodiments, the rigid polymer is an SAH peptide. In some embodiments, a single-stranded tail extends from the second terminal end of a rigid polymer or there is no extension from the second end of the rigid polymer. In some embodiments, the single-stranded tail at the second end of the rigid polymer is much shorter than the single-stranded tail that extend from the first end of the rigid polymer. In certain embodiments, a second terminal end of a rigid polymer (for example, the polymer itself or an attached single-stranded tail) comprises a reactive functional group for ligation onto the C- or N-terminus of the target protein or peptide using the methods described below.

The ability to perform site-specific modification of the termini of proteins is a very active area of research. In some embodiments, N-terminal specific modifications have been achieved by various methods, mainly exploiting the pKₐ differences between lysine (pKₐ˜10) and the N-terminal amine (pKₐ ˜8) (Chan, W.-K.; Ho, C.-M.; Wong, M.-K.; Che, C.-M., Oxidative amide synthesis and N-terminal α-amino group ligation of peptides in aqueous medium. Journal of the American Chemical Society 2006, 128 (46), 14796-14797; Chan, A. O.-Y.; Ho, C.-M.; Chong, H.-C.; Leung, Y.-C.; Huang, J.-S.; Wong, M.-K.; Che, C.-M., Modification of N-terminal α-amino groups of peptides and proteins using ketenes. Journal of the American Chemical Society 2012, 134 (5), 2589-2598; Kung, K. K.-Y.; Wong, K.-F.; Leung, K.-C.; Wong, M.-K., N-terminal α-amino group modification of peptides by an oxime formation-exchange reaction sequence. Chemical Communications 2013, 49 (61), 6888-6890; MacDonald, J. I.; Munch, H. K.; Moore, T.; Francis, M. B., One-step site-specific modification of native proteins with 2-pyridinecarboxyaldehydes. Nature chemical biology 2015, 11 (5), 326-331), with the N-terminal amine reacting via multiple methods including reductive alkylation (Chen, D.; Disotuar, M. M.; Xiong, X.; Wang, Y.; Chou, D. H.-C., Selective N-terminal functionalization of native peptides and proteins. Chem Sci 2017, 8 (4), 2717-2722), and installation of an azide by reaction with azidoacetic anhydride. (Biswas, S.; Song, W.; Borges, C.; Lindsay, S.; Zhang, P., Click addition of a DNA thread to the N-termini of peptides for their translocation through solid-state nanopores. ACS nano 2015, 9 (10), 9652-9664).

In some embodiments, attachment to the C-terminus can be carried out by enzymatic attachment of affinity tags using carboxypeptidase Y (Xu, G.; Shin, S. B. Y.; Jaffrey, S. R., Chemoenzymatic labeling of protein C-termini for positive selection of C-terminal peptides. ACS chemical biology 2011, 6 (10), 1015-1020), as well as by the differences in oxidation potential between the C-terminus and glutamate/aspartate, for single electron transfer reactions to alkylate the C-terminus. (Hoyt, E. A.; Cal, P. M.; Oliveira, B. L.; Bernardes, G. J., Contemporary approaches to site-selective protein modification. Nature Reviews Chemistry 2019, 3 (3), 147-171; Bloom, S.; Liu, C.; Kolmel, D. K.; Qiao, J. X.; Zhang, Y.; Poss, M. A.; Ewing, W. R.; MacMillan, D. W., Decarboxylative alkylation for site-selective bioconjugation of native proteins via oxidation potentials. Nature chemistry 2018, 10 (2), 205-211; Yu, Y.; Zhang, L.-K.; Buevich, A. V.; Li, G.; Tang, H.; Vachal, P.; Colletti, S. L.; Shi, Z.-C., Chemoselective peptide modification via photocatalytic tryptophan β-position conjugation. Journal of the American Chemical Society 2018, 140 (22), 6797-6800) In some embodiments, attachment of capture tags to the C-terminus can also be targeted to specific amino acid motifs (Tan, D. J. Y.; Cheong, V. V.; Lim, K. W.; Phan, A. T., A modular approach to enzymatic ligation of peptides and proteins with oligonucleotides. Chemical Communications 2021, 57 (45), 5507-5510) using sortase (Mao, H.; Hart, S. A.; Schink, A.; Pollok, B. A., Sortase-mediated protein ligation: a new method for protein engineering. J Am Chem Soc 2004, 126 (9), 2670-1), peptide asparaginyl ligase (Hemu, X.; El Sahili, A.; Hu, S.; Zhang, X.; Serra, A.; Goh, B. C.; Darwis, D. A.; Chen, M. W.; Sze, S. K.; Liu, C.-f.; Lescar, J.; Tam, J. P., Turning an Asparaginyl Endopeptidase into a Peptide Ligase. ACS Catalysis 2020, 10 (15), 8825-8834), or other enzymatic methods. (Nuijens, T.; Toplak, A.; Schmidt, M.; Ricci, A.; Cabri, W., Natural Occurring and Engineered Enzymes for Peptide Ligation and Cyclization. Frontiers in Chemistry 2019, 7 (829)).

In some embodiments, the C-terminus of the protein or peptide (modified with a capture tag) can be first captured by a first nanopore reader and held, followed by capture of the N-terminus (modified with a capture tag) by a second nanopore reader, or vice versa. After capture of one end in the first nanopore reader, the voltage can be reduced to hold the protein or peptide in place while the other end of the protein or peptide is captured in the second nanopore reader. After dual capture, the voltage can be increased to a high level (for example, +−220 mV) in both PLBs to denature the protein or peptide secondary structure and stretch it between the nanopore readers in the PLBs. In certain embodiments, the voltage may be controlled using the FPGA capture and control system to pass the peptide or protein between the two nanopore readers. As the protein or peptide passes through the nanopore readers, the sequence can be read via the blocking current levels, translocation time, and/or current noise of the amino acids within the readers. In certain embodiments, proteins and peptides can be multipassed to achieve the desired level of residue identification accuracy. In certain embodiments, proteins and peptides can be multipassed multiple times. In some embodiments, proteins and peptides can be multipassed between about 2 to about 1000 times, about 10 to about 100 times, or about 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 500, 550, 600, 650, 700, 750, 800, 850, 900, 950, 1000 times.

### Compositions

In certain embodiments, a composition comprises a target polymer comprising monomeric units, a first distal end and a second distal end, a capture tag at the first distal end and a capture tag at the second distal end of the target polymer. In some embodiments, a target polymer comprises a single-stranded RNA, a single-stranded DNA, a double-stranded DNA, an RNA/DNA heteroduplex, a protein or a peptide or any combination thereof. In certain embodiments, compositions can comprise target polymers and capture tags as described herein. In certain embodiments, methods of making compositions are as described herein.

### Tagged Polymer and Dual Nanopore Device Systems

In certain embodiments, a system comprises a tagged polymer and a dual nanopore device. In some embodiments, a system comprises a plurality of tagged polymers and a plurality of dual nanopore devices. In certain embodiments, a system comprises tagged polymers and dual nanopore devices as described herein. In certain embodiments, methods of using a system for sequencing tagged polymers and methods for associating tagged polymers with dual nanopore devices are as described herein.

### Dual Nanopore Sequencing Systems

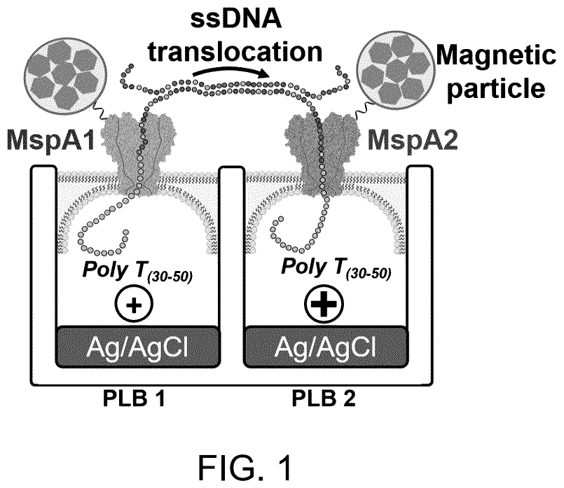

For nanopore based sequencing, a significant advantage may be derived from utilizing two adjacent nanopores (dual nanopores), rather than a single nanopore (see FIG. 1). With a dual nanopore setup, the speed of translocation, once the polymer molecule is captured by and across both readers, can be deliberately controlled by using opposing forces (i.e., electrophoresis, electroosmosis and/or pressure driven flow) at each nanopore to slowly thread the polymer through each of the pores. The opposing electrophoretic forces also hold the DNA taut, removing any secondary structure and allowing the signal to be due primarily to primary sequence. The strand can be flossed/multipassed back and forth through the nanopore to re-read the strand and improve accuracy. And, two nanopores allows for redundancy in sequencing, as base calling can be carried out at each nanopore.

FIG. 1 shows an example of capturing one strand of a double-stranded DNA strand across a dual biological nanopore sequencing system, by placing poly T single-stranded DNA tails onto one of the strands of the double-stranded DNA target. The complementary strand is mechanically unzipped from the captured strand, by electrophoretically driving the capture strand through the readers, i.e., the double-strand is too large to translocate through the readers and is therefore unzipped during translocation.

The system includes two biological nanopore readers magnetically positioned in individual planar lipid bilayers (PLBs) in close proximity (for example, about 10 nm to about 5 micrometers) to one another, each with its own high-speed field-programmable gate array (FPGA) controlled voltage biasing (see FIG. 1). A system can be utilized for (1) controllably capturing a target polymer across the two readers; (2) ensuring that the polymer is elongated/stretched through each reader (a requirement for nanopore-based strand sequencing); (3) controlling the rate and direction of strand translocation through the readers using voltage bias without the use of an enzyme/motor; (4) flossing/multipassing the polymer back and forth through the readers in order to re-read and improve sequencing accuracy; and (5) using two different readers simultaneously but independently for cross-validation, further improving the sequencing accuracy. Such system and methods have nanopore-based polymer sequencing applications, including, but not limited to, sequencing single-stranded DNA and RNA, double-stranded DNA and RNA, proteins and peptides.

The adjacent-dual-biological-nanopore-based sequencing platform described herein can combine the translocation control provided by dual-pore readers with the sequence sensitivity of biological nanopores to enable high-accuracy (>99.9%) sequencing. For example, systems described herein address the speed of polymer (for example, nucleic acid) translocation which compromises accurately resolution of individual monomers (for example, nucleotides) and the noise associated with strand compression and relaxation during free translocation that prohibits accurate reading of individual monomers (for example, nucleotides) based on the uncertain position of the strand at any given moment. The systems and methods described herein also avoid the limitations of enzymes/motors. Consequently, and significantly advantageous, systems described herein allow simplistic library preparation procedures that do not introduce artifacts/biases.

Without being limited by theory, the dual-pore system described herein induces a counter balance mechanism to control the rate of translocation through the readers. Here, one nanopore reader with its electrode and high-speed biasing mechanism functions as the “motor/brakes” or translocation control by pulling the polymer against the other nanopore reader and its electrode and high-speed biasing mechanism. The net translocation direction and speed are controlled via the ability to independently bias across each reader and have a net translocation force on the polymer through one of the readers, pulling against the other reader.

The innovations behind the platform and methodology are numerous, including (1) the utilization of two adjacent chip-based PLBs whose width narrows as their proximity to one another also decreases; (2) the use of a magnetic field across those PLBs to pull individual nanopore readers tagged with magnetic particles into close proximity of one other; (3) the utilization of voltage induced protein/PLB incorporation to insert one reader type into one PLB and another reader type into an adjacent PLB on the same chip/platform; and (4) the utilization FPGA logic in combination with high-speed DC voltage bias switching to semi-automate the capture of target polymer into the two adjacent biological readers and control the direction and rate of translocation, including the ability to floss/multipass the polymer back and forth through the readers in order to re-read the sequence or portion of the sequence of the polymer.

Magnetophoretic Migration of Nanopores Tagged with Magnetic Nanoparticles

In order to bring the two biological nanopore readers (for example, α-hemolysin and MspA) within a few hundred nanometers from each other within separate but adjacent PLBs, each biological nanopore reader can be tagged with a single magnetic nanoparticle. The biological nanopore readers then can be guided in the lipid bilayers to their appropriate measurement position using an external magnetic field. Magnetic nanoparticles are widely used for biomolecule separations, where permanent magnets can induce rapid migration of magnetic nanoparticles over long distances (mm-cm). One of the most common are magnetite (Fe₃O₄) nanoparticles, which are available from multiple vendors with a variety of surface functionalizations.

Although bulk magnetite is ferrimagnetic and can adopt a permanent magnetic moment, the magnetic moment of nanocrystalline magnetite realigns randomly due to random thermal fluctuations. However, these nanoparticles are superparamagnetic, and they adopt strong magnetic moments when placed in an external magnetic field. Under magnetic fields greater than 0.5 T magnetite nanoparticles reach a maximum saturation magnetization of 50-90 Am² kg⁻¹.

This induced magnetic dipole moment is subject to a force in a magnetic field gradient proportional to the saturation magnetization of the dipole, Mₛ, the mass of the particle, m, and the magnetic field, B, gradient: F_{mag}=(Mₛm∇·)B. Since the magnitude of the magnetic dipole moment is proportional to the mass of the particle, larger particles experience stronger forces. For spherical magnetic nanoparticles in solution, the field gradient force induces migration of the particles, known as magnetophoresis. The force driving migration is proportional to the particle density, p (5.2 g cm⁻³ for magnetite), and the cube of the diameter, d:

$${F}_{mag} = ( {M}_{s} {ρ}^{π} / 6 {d}^{3} \cdot ∇ ) B \qquad Eq . 1$$

High magnetic fields (>0.5 T) and magnetic field gradients are needed to induce migration of magnetic particles. Simple permanent magnets in contact with sample vials can generate field gradients that vary from 10-100 Tm⁻¹, which are sufficient to concentrate large magnetic nanoparticles used for biomolecule separations. Stronger magnetic field gradients are needed to induce migration of smaller particles, which can be produced with magnets incorporated into thin cells that generate gradients up to 10³ T m⁻¹. Magnetic microstructures, such as magnetic tips and magnetic tweezers, in an external magnetic field can produce even higher local gradients in the 10³ to 10⁴ T m⁻¹ range. The component configured to apply a magnetic field across the wells and induce migration of magnetic particles can be an external magnet alone (for example, a rare-earth permanent magnet) or electromagnet coil. The component configured to apply a magnetic field across the wells can be an external magnet (for example, a permanent magnet or an electromagnet coil) with magnetic microstructures made of ferromagnetic, paramagnetic, or super paramagnetic materials with high relative permeabilities (for example, Ni, magnetite) near the microwells, which include but are not limited to magnet tips, magnetic tweezers, coils, or strips. The component is a magnetic probe consisting of a permanent magnet or electromagnet coil built into a sharp micro-scale probe that can be moved near the wells to apply a magnetic field.

Assuming a magnetic field in excess of 0.5 T and a gradient of 10³ Tm⁻¹, a 50 nm diameter ferrite nanoparticle would experience a magnetic migration force of ˜25 fN. When the particle begins to migrate, the magnetophoretic force is countered by viscous drag, F_{d}, on the particle, which can be estimated using Stoke's law:

$${F}_{d} = - 3 πη dv \qquad Eq . 2$$

Where η is the viscosity, and v is the migration velocity of the particle. Since the drag force increases linearly with the velocity, eventually the viscous drag will counteract the magnetophoretic forces and the particle will achieve terminal velocity. Equations 1 and 2 can be used to solve for the terminal velocity of a particle, vₜ, when F_{mag}=−F_{d}:

$${v}_{t} = ( {M}_{s} ρ {d}^{2} \cdot ∇ ) B / 18 η \qquad Eq . 3$$

Under the influence of the magnetic field gradient described above, a 50 nm diameter particle would reach a terminal velocity of 50 μm s⁻¹, allowing it to traverse a 3 μm pore in 120 ms.

However, a protein nanopore also experiences drag due to the high viscosity of phospholipid membranes (0.1 Pa s) compared to water (0.001 Pa s), which slows the magnetophoretic migration. A pore-forming protein, for example, alpha hemolysin, with a radius of ˜10 nm migrating in a lipid bilayer would experience 7-fold higher drag forces than the 50 nm magnetic particle in aqueous solution, reduces the migration velocity to 7 μm s⁻¹. Even with the combined drag, the nanopore can still traverse the membrane in less than a second, allowing rapid focusing of the nanopores into the constriction region.

Although strong enough to induce migration, the magnetic forces cannot pull the protein nanopores out of the lipid membrane. The force required to remove hydrophobic peptides and proteins from phospholipid bilayers has been extensively studied with atomic force microscopy. These forces, in the 30-200 pN range, are 3 orders of magnitude larger than the magnetic force on the nanoparticles, indicating that the nanoparticle manipulation will not induce desorption of proteins from the bilayer.

These magnetic forces must also overcome random thermal diffusion to confine the nanopores to the constriction region of the well. The magnitude of concentrating effect of the magnetic forces can be modeled using a Boltzmann distribution based on the ratio of the magnetic potential energy along the membrane to the thermal energy. For example, a 6 μm long membrane-spanning well with the magnetic field gradient can be oriented in the y-axis. The spatial distribution of nanopore excess concentration, C, in the membrane well can be determined from the magnetic potential energy distribution, U_{mag}(x,y):

$$C ( x , y ) = {e}^{Umag ( x , y ) γ kT} \qquad Eq . 4$$

The magnetic field gradient is constant and aligned with the vertical axis, y, and there is no gradient in the horizontal axis, x. The potential energy distribution is the magnetic force multiplied by the distance, I, from the distal end of the membrane: U_{mag}(x,y)=−F_{mag}(l−y). The excess concentration profile for 30 and 50 nm magnetic nanoparticles in a magnetic field gradient of 1000 T m⁻¹ can be determined. These profiles show a high concentration of particles near the constriction region, and that the trapping energetics increase with the particle volume. However, the area of the membrane decreases as the membrane tapers to the constriction zone, which increases the energy needed to concentrate the nanopores. A probability distribution for locating nanopores (scaled by area) along the magnetic field gradient can be determined. Based on this model, nanopores tethered to 100 nm particles are confined to a 90 nm region in the constriction zone with 99% certainty. The confinement region, and thus the average spacing between the two protein nanopores, can be tuned by changing the magnetic field gradient, and/or the size of the magnetic nanoparticles.

Experiments have demonstrated that simple permanent magnets are capable of inducing migration of magnetic nanoparticles in viscous media. In mixtures of glycerol and water with a viscosity chosen to simulate the drag of a magnetic nanoparticle-tethered biological nanopore reader, small 50 nm magnetic particles can be induced to migrate and concentrated into a confined region near a rare-earth permanent magnet. This result indicates that magnetophoresis can be used to move and confine magnetic particles in conditions similar to those in the dual PLB microwells.

### Nanopores and Readers

As used herein, the term “nanopore” includes a nanopore reader (biological or solid state), a biological nanopore, an artificial engineered nanopore, a DNA nanopore, a peptide nanopore and a solid state nanopore.

The biological nanopore reader utilized to sequence a single-stranded polymer (DNA or RNA), double-stranded polymer (DNA or RNA), protein or peptide can be any biological nanopore, ion channel, transmembrane protein or DNA nanopore suitable for strand sequencing applications, and be either the wild-type form of that nanopore, ion channel, or transmembrane protein or a mutated, engineered, and/or chemically modified form. The sensing zone of a nanopore reader (for example, αHL or MspA) can be mutated to both improve DNA translocation and have a single, sharp sensing zone that could resolve/sequence individual nucleotides. Using DNA strand trapping methods allowed various groups to relatively quickly perform this work, optimizing various nanopore readers, including αHL, MspA, and CsgG. Mutations could also target the overall charge, size, and three-dimensional shape of the nanopore, or could be made to alter the interaction with the membrane (i.e., to more/less easily insert into a membrane, to remain inserted in the membrane longer). A more aggressive truncation or insertion of several amino acids could remove or introduce a recognition site, enhance sensitivity, and impart selectivity of a desired analyte. Mutations could also involve the introduction of non-natural amino acids with unique side chains and functional properties. Oligomeric proteins could be synthesized as a single chain. For instance, a hemolysin, a heptamer, could have its seven subunits expressed as a single protein, with amino acid linkers introduced in between what were formerly separate subunits to connect them into a single chain that will fold into a functional nanopore. Various molecules can be added either through in vitro conjugation to the desired pore site or through fusion protein expression to enhance the nanopore performance.

Completely synthetic biological based nanopores or ion channels would also be suitable. Non-specific, non-inclusive examples of biological nanopore, ion channels, or transmembrane proteins which could be utilized include but are not limited to alpha-hemolysin (αHL), aerolysin, Mycobacterium smegmatis porin A (MspA), Escherichia coli CsgG, Cytolysin A (ClyA), outer membrane protein F (OmpF), modified or mutant forms of secretin, and Fragaceatoxin C (FraC). Non-inclusive examples of synthetic engineered biological nanopores include DNA nanopores (also referred to as “DNA-based nanopores” or “DNA origami nanopores”) and engineered peptide nanopores. Chemical crosslinking agents that covalently link the individual subunits or that tune the performance of the nanopore readers could be made.

Tagging Protein Pore Readers with Magnetic Nanoparticles

To induce migration in an external magnetic field, the biological nanopore readers can be tagged with a single magnetic nanoparticle via a polymer linker. Each reader can be engineered such that it contains a single attachment adapter for binding the above-described magnetic nanoparticles. Adapters can be added to engineered residues positioned on the cis side of the reader, and then can be conjugated with a compatible reactive or high-affinity binding group on a linker polymer. The opposite end of the linker polymer can contain an orthogonal reactive or affinity binding group for attachment to chemically modified magnetic nanoparticles. The orthogonal pair allowing for attachment to the magnetic nanoparticles could include (but is not limited to) biotin/streptavidin, epitope tags (for example, c-Myc or HA tags), 6-His/NiNTA, alkyne/azide, transcyclooctene/tetrazene, Snaptag/O⁶-benzylguanine or gold binding peptide/gold surface coating on magnetic nanoparticles. A process for non-limiting examples of biological nanopore readers, αHL and MspA, are described herein.

A first biological nanopore reader, alpha-hemolysin (αHL) is composed of seven monomer units that fold to form the heptameric protein pore. Wild-type αHL does not have any naturally occurring cysteine residues and its N- and C-termini are located on the cis-side of the pore so they both can be used to readily attach a flexible linker. Various residues in this region can be mutated to cysteine that can serve as an attachment point for maleimide-biotin or other bifunctional linkers to allow tethering to magnetic nanoparticles. In addition to a single cysteine mutant, an alternative strategy would be to link the adapter to surface exposed amino groups, either from lysine or the N-terminal amine. In this scenario, an NHS-ester would be used as the reactive group specific to the protein nanopore reader on the bifunctional linker.

To ensure that only a single linker is present per protein reader, only one of seven monomers may be selected for containing the reactive cysteine group. To facilitate the separation of heteroheptameric pores with a single linker, the αHL mutant subunit can be equipped with a polyaspartate (D8) tail on the C-terminus and a 6×-His tag (or other purification tags) for later purification. To prepare the heptamers, mutant αHL (M) and αHL monomers without cysteine (WT) can be mixed in different molar ratios (M: WT=1:6-4:3) and allowed to co-assemble on rabbit erythrocyte membranes (rRBCMs) or liposomes. Membranes then can be solubilized in SDS and the heptamers separated on SDS-PAGE due to increased migration of the monomers with the negatively charged D8 tail (αHL heptamers are stable in SDS unless heated). Heptamer pores containing only one mutant monomer (M₁WT₆) can be passively eluted from the polyacrylamide with water. Further analysis can be achieved by heating the proteins to 95° C. and separating dissociated subunits in a second analytical gel.

Generating a single linker per protein could be done by controlling the ratio of protein to linker. Excess amounts of protein will allow for a significant amount of single linker protein (removing the requirement for a single mutant subunit in the heteroheptamer). The protein can then be reacted with the orthogonally tagged magnetic beads, and the magnetic beads can be utilized to separate the tagged protein from the untagged protein that will be present since it was in excess for the reaction with the linker. Alternatively, a single linker could be attached to a magnetic nanoparticle first, then reacted with protein. Magnetic nanoparticles with a single linker are commercially available from Nanopartz (Loveland, CO), and the single linker can be achieved by manipulation of the stoichiometry, or by more sophisticated solid phase exchange reactions.

A second reader could be MspA which is a homo-octameric pore, composed of eight monomers each 184 amino acid in size. Mutations made to MspA have allowed for successful DNA translocation and single base discrimination, as well as immobilization of gold nanoparticles. Tags can be added to MspA, at the periplasmic loop 6 (residues 121-127) and fusion proteins with peptide linkers that linked 2 MspA monomers together (linking the N-terminus of one MspA monomer to the C-terminus of a second MspA monomer) to form a dimer, 4 dimers then assembled to form the functional oligomers. The same approach used with αHL to separate and select readers with a single cysteine can be applied to MspA, as MspA is also able to retain oligomeric assembly in SDS-PAGE. A single point mutation introducing cysteine can be made at the N-terminus located on the exterior of the cis side of the pore. MspA with a single cysteine can then also be reacted with maleimide-containing bifunctional linkers. Alternatively, other biological nanopore readers such as CsgG or OmpF can be similarly modified with cysteine and purification tags on the cis side of the pore and substituted for αHL or MspA.

A linker consisting of polyethylene glycol (PEG) or DNA with a cysteine-reactive functional group can then be reacted with the cysteine-modified nanopore readers. A maleimide-PEG (70-720)-biotin bifunctional linker with a length ˜25-200 nm can place the magnetic particles some distance away from the protein to avoid interference with DNA translocation and sequencing. A synthetic polymer linker has a length of about 25 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm 180 nm, 190 nm or 200 nm. An alternative to a synthetic polymer linker, the reader and nanoparticle can be linked via double-stranded DNA (about 75-600 bp, about 25-200 nm in length). The double-stranded DNA can have a length of about 25 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm 180 nm, 190 nm or 200 nm. The double-stranded DNA can be 75 bp. Single-stranded DNA (ssDNA) can similarly be synthesized with a terminal maleimide modification for ligation to cysteine residues. Alternatively, ssDNA has previously been covalently attached to a single monomer of αHL via a disulfide linkage within the heptameric pore to study DNA duplex formation. Briefly, 5′-thiol-modified DNA oligonucleotide with a hexamethylene linker can be activated with 2,2′-dipyridyl disulfide in order to form 5′-S-thiopyridyl oligonucleotide. αHL, MspA, or other biological nanopore readers containing a single cysteine mutation can then be reacted with activated 5′-S-thiopyridyl oligonucleotide. If a cysteine label on the nanopore reader is insufficient for attachment to the PEG or DNA linker, an unnatural amino acid can be incorporated into the biological nanopore reader, to convey bioorthogonal reactivity, such as click chemistry, inverse electron demand Diels-Alder cycloaddition and others. For click chemistry in particular, an alkyne or azide unnatural amino acid can be incorporated into the nanopore reader, then reacted with a DNA or PEG linker containing the corresponding azide or alkyne.

The other end of the linker can be attached to the magnetic nanoparticle using affinity tags or covalent coupling chemistry. Bifunctional linkers with a cysteine-reactive group described above, and a biotin group on the opposite end can be attached to streptavidin coated magnetic particles (Ocean Nanotech, San Diego, CA) via the strong biotin streptavidin interaction. Other affinity tags, including FLAG and Myc, and digoxigenin could be used. A linker can also be covalently attached by reacting an amine-modified PEG linker directly with carboxylate-modified magnetic particles activated with an EDC reagent. For the DNA tethers, ssDNA complementary to the ssDNA attached to the nanopore reader can contain a terminal biotin-tag for attachment to streptavidin-coated magnetic nanoparticles. The oligonucleotide-modified readers can then hybridize with complementary strands attached to magnetic nanoparticles to link them together. The alternative affinity and covalent attachment methods described above can also be used to link ssDNA to biological nanopore readers or magnetic particles.

After isolating the assembled readers containing a single cysteine-conjugated linker, a population with a single magnetic particle often is purified. To minimize the number of particles tagged with multiple readers, the readers with reactive linkers can be incubated with an excess of about 5-500 nm magnetic nanoparticles coated with streptavidin (or other surface modifications). The unbound magnetic nanoparticles and reader-nanoparticle conjugates can be isolated from unbound readers by applying a magnetic field, followed by the purification of the reader-nanoparticle complex by Ni-NTA affinity chromatography (cysteine containing monomers will have a D8 tail and a 6×-His tag). Magnetic particles with no reader attached often are washed off with buffer, then immobilized assemblies are eluted with an imidazole gradient, separating particles that have one His-tagged reader from those that have multiple. Alternatively, single-tagged particles can be purified using a different tag to the biological nanopore reader (strepII or HA tag), with immobilized anti-αHL/anti-MspA antibodies, or via size exclusion chromatography.

While the description above describes the route and ability to tag or link a biological nanopore reader to a single magnetic particle, it should be specifically noted that multiple (about 2 to about 100) magnetic particles (about 5 to about 500 nm in diameter) could also be attached to a single reader, in order to be able to influence both their position via a magnetic field in bulk solution as well as within a planar lipid bilayer, black lipid membrane, triblock copolymer, etc. In any configuration, either using a single magnetic nanoparticle or multiple magnetic nanoparticles, these magnetic particles could be directly attached to the outside of the readers or tethered to the readers. Tethers can range in length from about 2.5 nm to about 1000 nm in length.

### Systems

The instrument described herein can include high-impedance, low-noise, amplifiers optimized for measuring low-level currents. An amplifier with independent, high-speed, DC bias often is incorporated for each reader electrode. Two amplifiers and two DC bias levels often are incorporated to measure and control a pair of coupled sensors. The amplifiers often are electrically connected to the reader chip which includes the coupled sensors (see next section). The signals often are filtered for protection from aliasing and digitized. An FPGA often is utilized to run a control protocol for the DC bias levels while a host computer archives and analyzes the data as well as providing high-level control of the instrument.

### Chips

The coupled sensors are sometimes fabricated on a fused quartz/silica substrate that is approximately 0.5 mm thick, but other high-resistivity, low-loss substrates are also appropriate to limit parasitic capacitance (for example, glass or sapphire). The substrate thickness can be flexible and can be about 0.1 mm to about 2.0 mm. A substrate could have a thickness of about 0.1 mm, 0.15 mm, 0.2 mm, 0.25 mm, 0.30 mm, 0.35 mm, 0.40 mm, 0.45 mm, 0.50 mm, 0.55 mm, 0.60 mm, 0.65 mm, 0.70 mm, 0.75 mm, 0.80 mm, 0.85 mm, 0.90 mm, 0.95 mm, 1.00 mm, 1.10 mm, 1.15 mm, 1.20 mm, 1.25 mm, 1.30 mm, 1.35 mm, 1.40 mm, 1.45 mm, 1.50 mm, 1.55 mm, 1.60 mm, 1.65 mm, 1.70 mm, 1.75 mm, 1.80 mm, 1.85 mm, 1.90 mm, 1.95 mm or 2.00 mm. The substrate with electrodes, contacts, interconnects, and insulative layer is referred to as a chip. Sometimes a substrate is a base structure of a chip onto which a chip is built. A chip can have a single coupled sensor site or thousands of coupled sensor sites. A coupled sensor site can include two electrodes comprised of metals such as Pt, Au, or Ag in close proximity to each other, for example, about 100 nm to about 10,000 nm. Electrodes are at a distance of about 100 nm, 200 nm, 300 nm, 400 nm, 500 nm, 600 nm, 700 nm, 800 nm, 900 nm, 1000 nm or up to 10,000 nm from each other. One or multiple larger electrodes may be included to form reference electrodes for the bath, but COTS external reference electrodes can be utilized. Variants with multiple reference electrodes may include different metals such as one electrode Pt and the other Ag for example. The Ag electrode variant chips may be treated to form stable Ag/AgCl electrodes. Electrodes can be connected to contact pads comprising Au or Pt on the periphery of the chip to connect the chip to the measurement system. A chip can be covered with about a 1-100 micrometer thick polymer insulator, such as SU-8, polyimide, parylene, or PTFE but other coatings that meet the specific requirements of chemical compatibility, insulative, and low-loss that are acceptable. A polymer insulator can have a thickness of about 1 μm, 2 μm, 5 μm, 10 μm, 20 μm, 30 μm, 40 μm, 50 μm, 60 μm, 70 μm, 80 μm, 90 μm, or 100 μm. An insulative layer can be patterned and etched in a manner to expose all the electrodes and contacts, thus creating openings over the electrodes and access to the contact pads. The openings can be then enlarged using thin-film processes, for example etching or ion milling, such that for a given coupled sensor site the minimum distance between the perimeter of one well to the perimeter of another well be about 10 nm to about 10,000 nm. A distance between the perimeter of one well to the perimeter of another well can be about 10 nm to about 1000 nm. A distance between the perimeter of one well to the perimeter of another well can be about 10 nm, 50 nm, 100 nm, 200 nm, 300 nm, 400 nm, 500 nm, 600 nm, 700 nm, 800 nm, 900 nm, 1000 nm, 2000 nm, or 10,000 nm. These openings define the shape of the wells or PLB cavity shape.

The shape of the PLB cavity is not arbitrary and operates in conjunction with an applied magnetic field to guide the reader pore to the required measurement site. For example, the shape of a PLB cavity can have a tilted snowcone shaped PLB cavity structure. The logic behind this concept is that the wider region having a 6 micrometers wide dimension, for example, provides a large insertion area for the reader pores whereas the smaller region having a 400 nm dimension, for example, confines the reader pore to the measurement area, i.e., the DNA capture zone. A dimension for the smaller region can be fabrication process and bilayer formation dependent. A dimension for the smaller region can be about 50 nm to about 1000 nm. A dimension for a smaller region is about 50 nm, 100 nm, 150 nm, 200 nm, 250 nm, 300 nm, 350 nm, 400 nm, 450 nm, 500 nm or 1000 nm. A dimension for a smaller region is about 50 nm, 100 nm, 150 nm, 200 nm or 250 nm. This concept also allows larger electrodes to be utilized, which last longer for Ag/AgCl electrodes and have a lower impedance.

This concept also allows large long-lasting electrodes to be formed and utilized to apply a magnetic field to the structure to produce a field gradient. The forces acting on a magnetic nanoparticle tethered to a protein nanopore in a lipid membrane that determine the terminal velocity: vₜ: nanopore-bilayer viscous drag, F_{d, pore}, particle-solution viscous drag, F_{d, part}, and magnetophoretic force, F_{mag}. The gradient in the magnetic field is then coupled to the magnetic nanoparticle tagged reader pore to pull the reader pore into the narrow section of the snowcone shaped PLB, using the shape of the PLB cavity as a guide. Application of the magnetic field can drive the two reader pores (i.e., one pore in each well) to within about 10 nm to about 5 μm of each other. Application of the magnetic field can drive the two reader pores (i.e., one pore in each well) to within a few hundred nanometers of each other. Application of the magnetic field can drive the two reader pores (i.e., one pore in each well) to within about 10 nm, 50 nm, 100 nm, 200 nm, 300 nm, 400 nm, 500 nm, or 1000 nm of each other. Other cavity shapes can be utilized, such as shapes that include a region having a larger surface area than another region within the shape. Non-limiting examples include football, triangular, diamond, crescent, oval, lightbulb, skull, and pill shapes. Cavity shapes include snowcone, football, teardrop, bullet, triangle, curvilinear triangle, crescent, circle, oval, ellipse, parabola, hyperbola, annulus, lens, circular segment, circular sector, heart, trefoil, quatrefoil, lightbulb, skull, pill, polygon, quadrilateral, star, diamond, trapezoid, square or rectangle. A well configuration can have a major length, major width, minor width, and angle (theta) between the virtual major length axis of each well. The two adjacent cavities or wells often are tilted towards one another. This tilted configuration permits a first region in one well having a smaller surface area, or the smallest surface area, to be located in close proximity to a second region in the other well having a smaller surface area, or the smallest surface area. Each well can be viewed as having a major (long) virtual axis parallel to the major length of the well. For wells that are tilted towards one another the long virtual axis of a first well is at an angle between about 2 degrees and about 170 degrees with respect to the long virtual axis of the second well, where an angle of 0 degrees is defined as the long virtual axis of each well-being parallel to one another and an angle of 90 degrees is defined as the long virtual axis of each well-being perpendicular to one another. The long virtual axis of a first well can be at an angle of about 2 degrees, 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 105 degrees, 110 degrees, 115 degrees or 120 degrees with respect to the long virtual axis of the second well. The long virtual axis of a first well can be at an angle of about 5 degrees to about 120 degrees with respect to the long virtual axis of the second well.

The well opening shape and the tilt of the wells often are selected such that the width of the opening of each well decreases as the distance between the wells decreases. The shape of the first well and the shape of the second well can be the same shape or a symmetric shape. The shape of the first well and the shape of the second well can be different.

The chip may also include thin film magnetic microstructure circuits to concentrate the flux around the cavities and generate large magnetic field gradients in localized regions. The magnetic circuits may be fabricated on a second chip that is either bonded to the sensor chip or placed below it in the test fixture. The magnetic microstructures can be fabricated using thin film processes and materials with high relative permeabilities such as but not limited to Ni, Fe, or Co. These structures can be made small (about 1 to about 100 μm) and localized around the cavity/PLB using thin film processes and lithography. They may include long strips of magnetic material that span several or all coupled sensors on a chip (for example, a magnetic strip composed of Ni or other metals with high magnetic susceptibility with thickness and depth of 1-50 μm spans multiple PLB pairs and generates strong local magnetic field gradient induced by an external permanent or electromagnet) or magnetic structures localized to a specific coupled sensor pair (for example, magnetic tip structures composed of similar materials positioned near the constriction point of each PLB pair). The magnetic microstructure circuits can be energized with an electromagnet near the magnetic microstructure (for example, within a distance of about 1 mm, 2 mm, 5 mm, 10 mm, 15 mm, or 25 mm) or by bringing the core of the electromagnet into contact with the microstructure tabs. A permanent magnet (for example, rare-earth magnet) would similarly work for energizing the magnetic microstructure circuit. Due to the high magnetic field gradient generated near small paramagnetic structures, smaller electromagnet coils or permanent magnets can be used with these microstructures.

### Magnetic Field Application

An external magnetic field can be applied to induce magnetophoresis of magnetic nanoparticles. The magnetic field can be applied with an external permanent magnet (for example, a permanent rare earth magnet that translates into position in plane near the dual PLBs) or coil (for example, a current applied to an electromagnetic coil in a fixed position near the PLBs to induce a magnetic field), or thin film microstructured magnetic circuits (i.e., not a magnet but a high permeability material) or coils fabricated into a chip.

A chip reader instrument sometimes includes a permanent magnet that can be positioned a variable distance from the chip edge. The distance can be about 1 mm, 2 mm, 3 mm, 5 mm, 10 mm, 12.5 mm, 15 mm, 20 mm, 25 mm, 30 mm, 35 mm, 40 mm, 45 mm, 50 mm, 100 mm, 200 mm or 500 mm from the edge of the chip. This configuration allows for fine tuning of the field gradient. The instrument can also accommodate a variety of permanent magnet sizes as necessary. The magnets can be oriented horizontally to the side of the chip for a brute force approach or can be oriented below the chip for a more localized region of high gradient, for example. The field characteristics as generated by finite element analysis for a small permanent magnet underneath the chip are much larger compared to a more powerful magnet placed on the side of the chip. This is because the magnet underneath the chip couples better to the surface of the chip.

Alternatively, a wire coil electromagnet near the chip can be used to apply the external magnetic field. By applying current to the electromagnetic coil, the magnetic field can be toggled on, off, adjusted continuously, or modulated with an external signal to provide fine manipulation of magnetic particles. The electromagnet often includes a wire coil close to the chip reader apparatus on a fixed mount. The coil may be placed on the side or bottom of the chip reader device.

Another option is to fabricate a microstructure magnetic circuit out of thin sheets of high permeability material such as mu-metal or Metglas alloys. The sheets of material could be laser cut or chemically etched (Fotofab, LLC, Chicago, IL) with the desired pattern and placed below the chip, on top of the polymer surface, in between the substrate and polymer, or even bonded to the underside of the chip directly. Although features are not as small as with thin film processes, they may still be sufficient for features in the 25-500 μm range. Considering that the substrate can be about 0.5 mm thick the microstructure will couple strongly to the PLB cavity. The microstructures can be energized with an electromagnet by bringing the core of the electromagnet into contact with the microstructure as mentioned previously.

### FPGA Capture and Control

In order to sequence a polymer on the adjacent-dual-biological-nanopore-based sequencing platform, a nanopore reader is associated with each well of the chip. A reader can be associated with each well of the chip using the following non-limiting example of a manufacturing process. The chip is initially bathed within an electrolyte solution or bath. A PLB then is formed over each PLB cavity on the chip, using known methods to paint or cast thin films of membrane-forming materials over the PLB cavity. The individual nanopore readers utilized then are inserted into each target PLB, utilizing a voltage cycling process (see U.S. Pat. No. 8,968,539). Any configuration of biological nanopore readers can be used for sequencing. For example, two αHL pores, two MspA pores, αHL pore plus MspA pore, or MspA pore plus CsgG pore may be utilized, with typically one pore in one PLB in one well. The nanopore readers can be of the same type, different types or the same type with one reader being wild-type and the other reader mutated, engineered, and/or chemically modified or both readers mutated, engineered, and/or chemically modified differently. The combination of readers will depend on the information that is desired from the sequencing application, and the combinations of readers could be chosen to provide the most complementary data on the polymer that is being sequenced.

Once the individual readers are present within each PLB, a magnetic field is applied to the chip in order to draw each magnetic nanoparticle tagged reader pore into close proximity of one other. An overview of this example of the insertion of magnetic particle tagged readers into PLBs and manipulation with a magnetic field to result in migration into the dual capture zone, may include: microwells photopatterned into SU-8 photoresist with Ag/AgCl electrodes in each well (for example, wells can be in a 3-20 μm deep SU-8 layer with the bottom of the well backfilled with Ag/AgCl), formation of planar supported lipid bilayer over each well using a magnetic stir bar or lipid painting method, capture of particle-tagged αHL into PLB1, capture of particle-tagged MspA into PLB2, and a magnet moved into position to generate high magnetic field gradient to move magnetic particle-tagged readers into dual-capture zone of PLBs.

A protocol for construction of a dual biological nanopore reader device may include: microwells photopatterned into SU-8 photoresist with Ag/AgCl electrodes in each well (for example, wells can be in a 3-20 μm deep SU-8 layer with the bottom of the well backfilled with Ag/AgCl), formation of planar supported lipid bilayer over each well using a magnetic stir bar or lipid painting method, application of high voltage bias (100-300 mV) to allow insertion of particle-tagged αHL into PLB1, application of voltage bias for insertion of particle-tagged MspA into PLB2 and a magnet moved into position to generate high magnetic field gradient to move magnetic particle-tagged readers into dual-capture zone of PLBs.

While a PLB is referred to here over each well, any suitable seal capable of retaining an individual reader can be utilized, including but not limited to a seal comprising phospholipids (for example, DPhPC, POPC, DOPC, DMPC, DoPhPC), surfactants, fatty acids (for example, mycolic acid), di-block copolymers (for example, polybutadiene-polyethylene oxide), tri-block copolymers (for example, poly-2-methyl-2-oxazoline-polydimethylsiloxane-poly-2-methyl-2-oxazoline), or polymerizable versions thereof.

After the reader pores have been inserted and have been magnetically moved to their measurement sites the DNA, RNA, protein or peptide sample often is added to the bath. A FPGA often is used to control the capture protocol, for example:
1. Set bias with respect to the bath reference electrode on one of the PLB electrodes (PLB1) to a positive level (for example, +100 mV to +250 mV) in order to capture one end of the polymer, while the other PLB electrode (PLB2) is set near zero or negative (for example,-60 mV to +60 mV) to prevent entry.
2. Monitor the current on PLB1 for capture of the single stranded polymer which will be registered by a decrease the current.
3. Once captured the bias protocol is reversed whereas PLB1 bias level is set to near zero or negative (for example,-60 mV to +60 mV) to prevent rapid translocation into well 1, and PLB2 is set to a positive level (for example, +100 mV to +250 mV) in order capture the free uncaptured end of the polymer into well 2.
4. Monitor the current on PLB2 for capture of the free end of the single stranded polymer which will be registered by a decrease the current.
5. Once captured both bias levels can be set to the same or similar low voltage (for example,-150 mV to +150 mV) to prevent escape of the polymer.

With both ends of the polymer captured the FPGA can run the measurement protocol to thread the polymer back and forth through the two reader pores. This will be done by setting both the bias on PLB1 and PLB2 with respect to the bath reference such that one acts as the drive voltage and the other functions as a “brake,” i.e., driving both captured ends of the polymer into each well, with one directionality overcoming the other. For example, if both electrodes are set to ˜+100 mV, based on the translocation kinetics of the utilized readers, the polymer will be quasi-stationary (because of DC offsets on the electrodes). However, if the PLB1 electrode is set to +100 mV and the PLB2 electrode is set to +50 mV there will be a net force pulling the polymer into the PLB1 cavity. The current measured from the PLB1 cavity will be with respect to the +100 mV bias but the translocation through the reader pore will be slower than a free polymer with +100 mV bias because PLB2 is pulling back on the polymer. Once the polymer is driven through one of the readers against the applied bias pulling the polymer in the direction of the other reader, and sequenced, before the polymer is fully pulled out and escapes from that other reader, the FPGA can trigger a reversal of the applied biases to drive the polymer back through that other reader, against the applied bias pulling the polymer in the direction of the reader that it was originally driven through. This would enable the polymer to be first sequenced though one of the readers and then sequenced through the other reader, while maintaining capture of the polymer via both readers. This multipassing or flossing strategy, all while maintaining capture of the polymer by both adjacent readers, can be carried out between 2 to 10,000 times. Multipassing can be 2, 3, 5, 10, 20, 50, or 100 times, with the number of multipasses increasing in a linear, quadratic, or exponential manner in order to make replicate measurements and improve the quality of the sequencing data to a desired level by for example, increasing base calling accuracy, decreasing error rates or deletions or insertions, improving detection of modified nucleobases.

During any of these voltage control steps an applied bias of −1 V to 1 V could be utilized, with an optimal voltage range being about −220 mV to about +220 mV. The voltage range can be about −220 mv, ˜210 mv, ˜200 mv, ˜190 mv, ˜180 mv, ˜170 mv,-160 mv, ˜150 mv, ˜140 mv, ˜120 mv, ˜100 mv, ˜90 mv, ˜80 mv, ˜70 mv, ˜60 mv, ˜50 mv, ˜40 mv, ˜30 mv, ˜20 mv, ˜10 mv, +10 mv, +20 mv, +30 mv, +40 mv, +50 mv, +60 mv, +70 mv, +80 mv, +90 mv, +100 mv, +110 mv, +120 mv, +130 mv, +140 mv, +150 mv, +160 mv, +170 mv, +180 mv, +190 mv, +200 mv, +210 mv or +220 mv, with the magnitude of the voltage being used to control the tension of the captured polymer and the difference in the voltage between the PLBs controlling the translocation velocity.

Alternative versions of this dual polymer strand capture and sequencing include:
A. Setting both electrode biases to positive and capturing one end of the polymer via one reader and the other end of the polymer via the other reader simultaneously or in a similar timeframe, and then setting the biases to control the rate and directionality of strand translocation.
B. Driving the polymer all the way into one of the wells through one of the readers and then driving it out and capturing the end that exits via the other reader.

Triggers that can be used to automatically or semi-automatically switch the applied biases and drive the polymer in the opposite direction inducing multipassing or flossing of the polymer through the two readers include, but are not limited to:
I. Set periods of time
II. Exit signatures of the end of the polymer coming out of one of the readers, before fully escaping out into bulk
III. Specific sequences of the polymer
IV. Changes in ionic current noise (for example, root mean square deviation, skew in the distribution)
V. Molecular markers added to the polymer at specific or arbitrary locations in the form of adducts

It may be appropriate to use the current signature of the reader, to sequence the polymer, as the polymer is translocating through the reader into the well in which that reader resides. It may be appropriate to use the current signature of the reader, to sequence the polymer, as the polymer is translocating back out of the reader, out of the well in which that reader resides, in the direction of the other reader or well. It may be appropriate to sequence the polymer in both translocation directions through both readers or any combination thereof. Polymer sequence determination from any of these translocation directions or configurations, as well as from multipass or flossing reads of the polymer, can then be used in combination with one another to determine the sequence of the polymer with a higher accuracy, than any single pass sequence determination would allow for individually.

It may be appropriate to sequence the polymer with a single pass. It may be appropriate to sequence the polymer with multiple passes.

It may be appropriate to capture the polymer with both adjacent readers and then drive and sequence the polymer all the way through one of the readers completely into the well in which that reader resides. Once fully captured in that well, one of the ends of the polymer can be translocated back out of the well and that end of the polymer can be captured by the other reader, recapturing that polymer strand across both adjacent readers. The polymer strand could then be driven and sequenced all the way through the other reader completely into the other well. This process could then be carried out multiple times: capture of the polymer with both readers, driving and sequencing the polymer all the way through one of the readers, capture of the polymer with both readers, driving and sequencing the polymer all the way through the other reader, repeat.

### Products of Manufacture and Kits

Provided are products of manufacture and kits for practicing methods as provided herein; and optionally, products of manufacture and kits can further comprise instructions for practicing methods as provided herein. In alternative embodiments, products of manufacture and kits as provided herein have contained therein, or comprise, a system as provided herein, wherein the system can comprise: a dual nanopore device; and a tagged target polymer comprising a capture tag at a first distal end and a capture tag at a second distal end, wherein the capture tag comprises a single-stranded tail.

In alternative embodiments, products of manufacture and kits as provided herein have contained therein, or comprise, a capture tag as provided herein, for example, a capture tag comprising a double-stranded DNA segment comprising a first end, a second end, a capture strand and a complementary strand, wherein: at the first end of the double-stranded DNA segment a single-stranded tail extends from the capture strand and an overhang or a single-stranded tail extends from the complementary strand, or there is no extension from the complementary strand; and, at the second end of the double-stranded DNA segment the capture strand or the capture strand and the complementary strand are configured to attach to a target polymer.

The invention will be further described with reference to the examples described herein; however, it is to be understood that the invention is not limited to such examples.

### EXAMPLES

The examples set forth below illustrate certain implementations and do not limit the technology.

### Example 1: Use of Capture Tags

Preliminary experiments with long (>500 nucleotides), single-stranded DNA (ssDNA) has shown an extremely low capture rate by various biological nanopores, under standard experimental conditions, with almost no translocation events observed within a reasonable time frame (tens of minutes), likely due to the extensive secondary structure and inaccessible ends of the DNA.

One way to improve the capture frequency of a very long ssDNA is to modify the 5′ and/or 3′ end of the strand by adding a capture tag consisting of a DNA duplex followed by a single-stranded tail to facilitate entry into the pores. The 500 nucleotide ssDNA target was also synthesized with an additional poly-A tail (about 30-50 nucleotides) or poly T tail (about 30 to about 50 nucleotides) on each end, and annealed to a short DNA oligonucleotide to form a duplex (about 15 to about 20 nucleotides) followed by single poly-A (about 30 to about 50 nucleotides) adjacent to the tail segments (FIG. 2 top 3 panels). Without capture tags, the ssDNA could not be captured by nanopore readers. After modifying the ssDNA with the duplex sequence and the single-stranded tails, the target ssDNA could be captured and translocated through the nanopore reader at a high rate, resulting in an event every ˜11 s when analyzing 25 nM DNA in solution (kₒₙ=3.5×10⁶ M⁻¹s⁻¹).

### Example 2: Listing of Certain Embodiments

Provided hereafter is a listing of certain non-limiting examples of embodiments of the technology.

A1. A method for associating a target polymer with dual nanopores, comprising:
(a) providing a target polymer comprising monomeric units, a first distal end, a second distal end, a capture tag at the first distal end and a capture tag at the second distal end, thereby providing a tagged polymer;
(b) providing a first nanopore and a second nanopore;
(c) driving the first distal end of the tagged polymer through the first nanopore, thereby capturing the first distal end of the tagged polymer by the first nanopore; and
(d) driving the second distal end of the tagged polymer through the second nanopore, thereby capturing the second distal end of the tagged polymer by the second nanopore.

A1.1. The method of embodiment A1, wherein the tagged polymer is in a fluid and the fluid containing the tagged polymer is contacted with the first nanopore and the second nanopore.

A2. The method of embodiments A1 or A1.1, wherein driving the first distal end of the tagged polymer through the first nanopore and/or driving the second distal end of the tagged polymer through the second nanopore comprises electrophoretic control and/or electroosmotic control and/or pressure driven flow.

A3. The method of any one of embodiments A1-A2, wherein steps (c) and (d) comprise FPGA controlled dual capture.

A4. The method of any one of embodiments A1-A3, wherein after step (d), electrophoretically and/or electroosmotically driving at least a portion of the tagged polymer through the first nanopore or at least a portion of the tagged polymer through the second nanopore; and identifying monomeric units of the tagged polymer as the tagged polymer translocates through the first nanopore or the second nanopore, thereby determining the sequence of at least a portion of the target polymer.

A5. The method of embodiment A4, wherein identifying monomeric units comprises detecting a current signature, translocation time and/or associated current noise level modulation associated with each of the monomeric units that translocates through the first nanopore or the second nanopore.

A6. The method of any one of embodiments A1-A3, wherein
(e) after step (d), electrophoretically and/or electroosmotically driving at least a portion of the tagged polymer through the first nanopore;
(f) identifying monomeric units of the tagged polymer as the tagged polymer translocates through the first nanopore, thereby determining the sequence of at least a portion of the target polymer;
(g) reversing the voltage bias between the first nanopore and the second nanopore;
(h) electrophoretically and/or electroosmotically driving at least a portion of the tagged polymer through the second nanopore;
(i) identifying monomeric units of the tagged polymer as the tagged polymer translocates through the second nanopore, thereby determining the sequence of at least a portion of the target polymer;
(j) reversing the voltage bias between the first nanopore and the second nanopore;
(k) electrophoretically and/or electroosmotically driving at least a portion of the tagged polymer through the first nanopore;
(l) identifying monomeric units of the tagged polymer as the tagged polymer translocates through the first nanopore, thereby determining the sequence of at least a portion of the target polymer; and
(m) repeating steps (g) to (l) to re-read the sequence or portion of the sequence of the target polymer.

A7. The method of embodiment A6, wherein identifying monomeric units comprises detecting a current signature, translocation time and/or associated current noise level modulation associated with each of the monomeric units that translocates through the first nanopore or the second nanopore.

A8. The method of any one of embodiments A1-A7, wherein the first nanopore and the second nanopore are part of a dual nanopore device.

A8.1. The method of embodiment A8, wherein the dual nanopore device comprises:
a chip;
a first well disposed adjacent to a second well on the chip;
a first seal over the first well and a second seal over the second well, and
a first nanopore is in the first seal and a second nanopore is in the second seal.

A9. The method of embodiment A8 or A8.1, wherein each nanopore independently is tethered to one or more magnetic particles.

A10. The method of embodiment A8.1 or A9, wherein each seal of the device independently comprises a planar lipid bilayer, surfactant bilayer, diblock copolymer bilayer or triblock copolymer monolayer.

A11. The method of any one of embodiments A1-A10, wherein each nanopore comprises a biological nanopore.

A12. The method of embodiment A11, wherein each biological nanopore independently is chosen from alpha-hemolysin (αHL), aerolysin, Mycobacterium smegmatis porin A (MspA), Escherichia coli CsgG, cytolysin A (ClyA), outer membrane protein F (OmpF), modified or mutant forms of secretin, or Fragaceatoxin C (FraC).

A13. The method in any one of the embodiments A1-A10, wherein each nanopore comprises an engineered DNA or peptide nanopore.

A14. The method of embodiment A13, wherein the engineered DNA nanopore comprises a DNA tile or DNA origami nanopore.

A15. The method of any one of embodiments A1-A10, wherein each nanopore is a biological nanopore, an engineered DNA nanopore or a peptide nanopore.

A16. The method of any one of embodiments A1-A15, wherein the first and second nanopores are the same.

A17. The method of any one of embodiments A1-A15, wherein the first and second nanopores are different.

A18. The method of any one of embodiments A1-A17, wherein a capture tag comprises a single-stranded tail.

A18.1. The method of any one of embodiments A1-A18, wherein the target polymer is a target strand or the target polymer comprises a target strand.

A18.2. The method of embodiment A18.1, whereby the single-stranded tail enables capture of the target strand by the first nanopore and the second nanopore.

A18.3. The method of any one of embodiments A18-A18.2, wherein the single-stranded tail comprises nucleic acid.

A19. The method of embodiment A18.3, wherein the nucleic acid comprises DNA.

A20. The method of embodiment A18.3, wherein the nucleic acid comprises RNA.

A21. The method of embodiment A19, wherein the single-stranded nucleic acid tail comprises an unstructured DNA heteropolymer or an unstructured DNA homopolymer.

A22. The method of embodiment A19, wherein the single-stranded nucleic acid tail comprises a homopolymer of A, C or T, abasic sites or modified nucleobases.

A23. The method of embodiment A19, wherein the single-stranded nucleic acid tail comprises a non-self complementary, non-homopolymer sequence of more than one type of base, comprising combinations of A, C, T, G, or abasic sites which does not fold up onto itself.

A24. The method of embodiment A22, wherein the single-stranded nucleic acid tail comprises a poly A homopolymer.

A25. The method of any one of embodiments A18.3-A24, wherein the single-stranded nucleic acid tail comprises at least 20 nucleotides.

A25.1. The method of any one of embodiments A18.3-A24, wherein the single-stranded nucleic acid tail comprises about 20 to about 100 nucleotides.

A26. The method of any one of embodiments A18-A18.2, wherein the single-stranded tail comprises a charged polymer.

A26.1. The method of embodiment A26, wherein the charged polymer comprises a charged peptide polymer.

A26.2. The method of embodiment A26.1, wherein the charged peptide polymer comprises polyarginine, polylysine or polyglutamate.

A26.3. The method of any one of embodiments A18-A18.2, wherein the single-stranded tail comprises a synthetic polymer.

A26.4. The method of embodiment A26.3, wherein the synthetic polymer comprises polystyrene sulfonate, polyamine, polyacrylate or polyvinyl sulfonate.

A27. The method of any one of embodiments A18-A26.4, wherein the target polymer comprises a single-stranded tail at the first distal end and a single-stranded tail at the second distal end.

A27.1. The method of embodiment A27, wherein the single-stranded tail comprises a homopolymer.

A27.2. The method of embodiment A27.1, wherein the homopolymer is a poly A homopolymer.

A28. The method of any one of embodiments A1-A27.2, wherein a capture tag comprises a double-stranded DNA segment comprising:
a first end, a second end, a capture strand and a complementary strand;
at the first end of the double-stranded DNA segment a single-stranded tail extends from the capture strand and a non-complementary overhang or a single-stranded tail extends from the complementary strand or there is no extension from the complementary strand; and
at the second end of the double-stranded DNA segment the capture strand is attached to a target strand of the target polymer.

A29. The method of embodiment A28, wherein the double-stranded DNA segment comprises about 15 to about 1000 nucleotides.

A30. The method of embodiment A29, wherein the double-stranded DNA segment comprises about 15 to about 20 nucleotides.

A31. The method of any one of embodiments A28-A30, wherein a non-complementary overhang or a single-stranded tail extends from the complementary strand of the double-stranded DNA segment and the non-complementary overhang or the single-stranded tail is attached to a blocking molecule.

A32. The method of embodiment A31, wherein the blocking molecule comprises a biotin/streptavidin complex, an antigen/antibody complex, a nanoparticle, a bulky DNA or RNA structure, a G-quadruplex, an i-motif, a cruciform-forming sequence, a pseudoknot a triple helix, a dendrimer, a polysaccharide, polyethylene glycol, a gold nanoparticle or a polystyrene nanoparticle.

A33. The method of any one of embodiments A1-A32, wherein the capture tag at each end of a target polymer has the same composition and the same length.

A34. The method of any one of embodiments A1-A32, wherein the capture tag at each end of a target polymer has a different composition and/or a different length.

A35. The method of any one of embodiments A1-A34, wherein the target polymer comprises a nucleic acid.

A36. The method of embodiment A35, wherein the nucleic acid comprises DNA.

A37. The method of embodiment A36, wherein the DNA is single-stranded.

A38. The method of embodiment A37, wherein the single-stranded DNA comprises a plurality of annealed short complementary DNA sequences.

A38.1. The method of embodiment A38, wherein the short complementary DNA sequences comprise about 6 to about 10 nucleotides.

A39. The method of embodiment A36, wherein the DNA is double-stranded.

A40. The method of embodiment A35, wherein the nucleic acid comprises RNA.

A41. The method of embodiment A40, wherein the RNA comprises a plurality of annealed short complementary DNA sequences.

A41.1. The method of embodiment A41, wherein the short complementary DNA sequences comprise about 6 to about 10 nucleotides.

A42. The method of embodiment A40, wherein the RNA comprises a complementary strand of DNA.

A43. The method of any one of embodiments A35-A42, wherein the nucleic acid comprises at least 100 nucleotides.

A44. The method of any one of embodiments A1-A27.2, wherein the target polymer comprises a protein or a peptide.

A45. The method of embodiment A44, wherein the protein or peptide comprises greater than about 10 amino acids.

A46. The method of embodiment A44 or A45, wherein a capture tag comprises
a double-stranded DNA segment comprising:
   a first end, a second end, a capture strand and a complementary strand;
   at the first end of the double-stranded DNA segment a single-stranded tail extends from the capture strand and a non-complementary overhang or a single-stranded tail extends from the complementary strand or there is no extension from the complementary strand;
at the second end of the double-stranded segment a single-stranded tail extends from the capture strand or there is no extension from the capture strand, the capture strand is configured to attach to an N terminus or a C terminus of a protein or a peptide and a non-complementary overhang or a single-stranded tail extends from the complementary strand or there is no extension from the complementary strand.

A47. The method of embodiment A46, wherein the double-stranded DNA segment comprises about 1500 to about 10,000 nucleotides.

A47.1. The method of embodiment A46 or A47, wherein the double-stranded DNA segment at the N terminus and the C terminus of the protein or the peptide target have the same length.

A47.2. The method of embodiment A46 or A47, wherein the double-stranded DNA segment at the N terminus and the C terminus of the protein or the peptide target a different composition and/or a different length.

A48. The method of any one of embodiments A46-A47.2, wherein at the first and the second end of the double-stranded DNA segment a non-complementary overhang or a single-stranded tail extends from the complementary strand and the non-complementary overhang or the single-stranded tail is attached to a blocking molecule.

A49. The method of embodiment A48, wherein the blocking molecule comprises a biotin/streptavidin complex, an antigen/antibody complex, a nanoparticle, a bulky DNA or RNA structure, a G-quadruplex, an i-motif, a cruciform-forming sequence, a pseudoknot a triple helix, a dendrimer, a polysaccharide, polyethylene glycol, a gold nanoparticle or a polystyrene nanoparticle.

A50. The method of embodiment A44 or A45, wherein a capture tag comprises:
a rigid polymer comprising a first end and a second end;
a single-stranded tail extends from the first end of the rigid polymer;
a single-stranded tail extends from the second end of the rigid polymer or there is no extension from the second end of the rigid polymer; and
the second end of the rigid polymer is configured to attach to an N terminus or a C terminus of a protein or a peptide.

A51. The method of embodiment A50, wherein the rigid polymer at the N terminus and at the C terminus of the protein or the peptide target have the same length.

A52. The method of embodiment A50, wherein the rigid polymer at the N terminus and at the C terminus of the protein or the peptide target have a different composition and/or a different length.

A53. The method of any one of embodiments A50-A52, wherein the rigid polymer comprises a polypeptide.

A54. The method of embodiment A53, wherein the polypeptide is a single alpha-helix (SAH), a collagen-like helix or a coil-coil structure.

B1. A capture tag comprising:
a double-stranded DNA segment comprising
   a first and a second end; and
   a single-stranded tail attached to a capture strand of the double-stranded DNA segment at the first end.

B2. The capture tag of embodiment B1, wherein at the second end of the double-stranded DNA segment the capture strand is configured to attached to a target strand of a target polymer.

B3. The capture tag of embodiment B1 or B2, wherein the double-stranded DNA segment comprises about 15 to about 1000 nucleotides.

B3.1. The capture tag of embodiment B3, wherein the double-stranded DNA segment comprises about 15 to about 20 nucleotides.

B4. The capture tag of any one of embodiments B1-B3.1, wherein the double-stranded DNA segment comprises a complementary strand and at the first end of the double-stranded DNA segment the complementary strand comprises a non-complementary overhang or a single-stranded tail or there is no extension from the complementary strand.

B4.1 The capture tag of embodiment B4, wherein at the second end of the double-stranded DNA segment the complementary strand is configured to attached to a non-target strand of a target polymer.

B5. The capture tag of embodiment B4 or B4.1, wherein a non-complementary overhang or a single-stranded tail extends from the complementary strand and the non-complementary overhang or the single-stranded tail is attached to a blocking molecule.

B6. The capture tag of embodiment B5, wherein the blocking molecule comprises a biotin/streptavidin complex, an antigen/antibody complex, a nanoparticle, a bulky DNA or RNA structure, a G-quadruplex, an i-motif, a cruciform-forming sequence, a pseudoknot a triple helix, a dendrimer, a polysaccharide, polyethylene glycol, a gold nanoparticle or a polystyrene nanoparticle.

B7. The capture tag of any one of embodiments B1-B6, wherein the single-stranded tail comprises nucleic acid.

B8. The capture tag of embodiment B7, wherein the nucleic acid comprises DNA.

B9. The capture tag of embodiment B7, wherein the nucleic acid comprises RNA.

B10. The capture tag of embodiment B8, wherein the single-stranded nucleic acid tail comprises an unstructured DNA heteropolymer or an unstructured DNA homopolymer.

B11. The capture tag of embodiment B8, wherein the single-stranded nucleic acid tail comprises a homopolymer of A, C or T, abasic sites or modified nucleobases.

B12. The capture tag of embodiment B8, wherein the single-stranded nucleic acid tail comprises a non-self complementary, non-homopolymer sequence of more than one type of base, comprising combinations of A, C, T, G, or abasic sites which does not fold up onto itself.

B13. The capture tag of embodiment B11, wherein the single-stranded nucleic acid tail comprises a poly A homopolymer.

B14. The capture tag of any one of embodiments B7-B13, wherein the single-stranded nucleic acid tail comprises 20 or more nucleotides.

B15. The capture tag of any one of embodiments B7-B13, wherein the single-stranded nucleic acid tail comprises about 20 to about 100 nucleotides.

B16. The capture tag of any one of embodiments B1-B6, wherein the single-stranded tail comprises a charged polymer.

B17. The capture tag of embodiment B16, wherein the charged polymer comprises a charged peptide polymer.

B17.1. The capture tag of embodiment B17, wherein the charged peptide polymer comprises polyarginine, polylysine or polyglutamate.

B17.2. The capture tag of any one of embodiments B1-B6, the single-stranded tail comprises a synthetic polymer.

B17.3. The capture tag of embodiment B17.2, wherein the synthetic polymer comprises polystyrene sulfonate, polyamine, polyacrylate or polyvinyl sulfonate.

B18. A capture tag comprising a double-stranded DNA segment comprising a capture strand with a single-stranded tail attached at one end of the capture strand and the opposite end of the capture strand is configured to attach to a N terminus or a C terminus of a target protein or peptide.

B19. The capture tag of embodiment B18, wherein the end of the capture strand configured to attach to a protein or peptide comprises a reactive group.

B20. The capture tag of embodiment B18 or B19, wherein the double-stranded DNA comprises a complementary strand comprising a single-stranded tail attached at each end.

B21. The capture tag of embodiment B20, wherein the single-stranded tails are each attached to a blocking molecule.

B22. The capture tag of embodiment B21, wherein the blocking molecule comprises a biotin/streptavidin complex, an antigen/antibody complex, a nanoparticle, a bulky DNA or RNA structure, a G-quadruplex, an i-motif, a cruciform-forming sequence, a pseudoknot a triple helix, a dendrimer, a polysaccharide, polyethylene glycol, a gold nanoparticle or a polystyrene nanoparticle.

B23. A capture tag comprising:
a double-stranded DNA segment comprising:
   a first end, a second end, a capture strand and a complementary strand;
   at the first end of the double-stranded DNA segment a single-stranded tail extends from the capture strand and a non-complementary overhang or a single-stranded tail extends from the complementary strand or there is no extension from the complementary strand; and
   at the second end of the double-stranded segment a single-stranded tail extends from the capture strand or there is no extension from the capture strand, the capture strand is configured to attach to an N terminus or a C terminus of a protein or a peptide and a non-complementary overhang or a single-stranded tail extends from the complementary strand or there is no extension from the complementary strand.

B24. The capture tag of embodiment B23, wherein at the second end of the double-stranded DNA segment the capture strand comprises a reactive group.

B25. The composition of embodiment B23 or B24, wherein at the first and the second end of the double-stranded DNA segment a non-complementary overhang or a single-stranded tail extends from the complementary strand and the non-complementary overhang or the single-stranded tail is attached to a blocking molecule.

B26. The composition of embodiment B25, wherein the blocking molecule comprises a biotin/streptavidin complex, an antigen/antibody complex, a nanoparticle, a bulky DNA or RNA structure, a G-quadruplex, an i-motif, a cruciform-forming sequence, a pseudoknot a triple helix, a dendrimer, a polysaccharide, polyethylene glycol, a gold nanoparticle or a polystyrene nanoparticle.

B27. The capture tag of any one of embodiments B18-B26, wherein the double-stranded DNA segment comprises about 1000 to about 10,000 base pairs.

B28. The capture tag of embodiment B27, wherein the double-stranded DNA segment comprises about 1,500 to about 5,000 base pairs.

B29. The capture tag of any one of embodiments B18-B28, wherein the single-stranded tail comprises nucleic acid.

B30. The capture tag of embodiment B29, wherein the nucleic acid comprises DNA.

B31. The capture tag of embodiment B29, wherein the nucleic acid comprises RNA.

B32. The capture tag of embodiment B30, wherein the single-stranded nucleic acid tail comprises an unstructured DNA heteropolymer or an unstructured DNA homopolymer.

B33. The capture tag of embodiment B30, wherein the single-stranded nucleic acid tail comprises a homopolymer of A, C or T, abasic sites or modified nucleobases.

B34. The capture tag of embodiment B30, wherein the single-stranded nucleic acid tail comprises a non-self-complementary, non-homopolymer sequence of more than one type of base, comprising combinations of A, C, T, G, or abasic sites which does not fold up onto itself.

B35. The capture tag of embodiment B33, wherein the single-stranded nucleic acid tail comprises a poly A homopolymer.

B36. The capture tag of any one of embodiments B18-B35, wherein the single-stranded nucleic acid tail extending from the capture strand of the double-stranded DNA segment comprises about 20 to about 1000 nucleotides.

B37. The capture tag of embodiment B36, wherein the single-stranded nucleic acid tail comprises about 40 to about 100 nucleotides.

B38. The capture tag of any one of embodiments B18-B28, wherein the single-stranded tail comprises a charged polymer.

B39. The capture tag of embodiment B38, wherein the charged polymer comprises a charged peptide polymer.

B39.1. The capture tag of embodiment B39, wherein the charged peptide polymer comprises polyarginine, polylysine or polyglutamate.

B40. The capture tag of any one of embodiments B18-B28, wherein the single-stranded tail comprises a synthetic polymer.

B41. The capture tag of embodiment B40, wherein the synthetic polymer comprises polystyrene sulfonate, polyamine, polyacrylate or polyvinyl sulfonate.

B42. A capture tag comprising a rigid polymer comprising a single-stranded tail attached at one end and the opposite end is configured to attach to a N-terminus or a C-terminus of a protein or peptide.

B43. The capture tag of embodiment B42, wherein the end configured to attach to a protein or peptide comprises a reactive group.

B44. The capture tag of embodiment B42 or B43, wherein the rigid polymer comprises a polypeptide.

B45. The capture tag of embodiment B44 wherein the polypeptide is a single alpha-helix (SAH), a collagen-like helix or a coil-coil structure.

B46. The capture tag of embodiment B45, wherein the polypeptide is a single alpha-helix (SAH) comprising about 50 to about 3000 amino acids.

B47. A capture tag comprising:
a rigid polymer comprising a first end and a second end;
a single-stranded tail extends from the first end;
a single-stranded tail extends from the second end or there is no extension from the second end; and
the second end of the rigid polymer is configured to attach to an N terminus or a C terminus of a protein or a peptide.

B48. The capture tag of embodiment B47, wherein the second end of the rigid polymer comprises a reactive group.

B49. The capture tag of embodiment B47 or B48, wherein the rigid polymer comprises a polypeptide.

B50. The capture tag of embodiment B49, wherein the polypeptide is a single alpha-helix (SAH), a collagen-like helix or a coil-coil structure.

B51. The capture tag of embodiment B50, wherein the polypeptide is a single alpha-helix (SAH) comprising about 50 to about 3000 amino acids.

B52. The capture tag of any one of embodiments B42-B51, wherein the single-stranded tail comprises a nucleic acid.

B53. The capture tag of embodiment B52, wherein the nucleic acid comprises DNA.

B54. The capture tag of embodiment B52, wherein the nucleic acid comprises RNA.

B55. The capture tag of embodiment B53, wherein the single-stranded nucleic acid tail comprises an unstructured DNA heteropolymer or an unstructured DNA homopolymer.

B56. The capture tag of embodiment B53, wherein the single-stranded nucleic acid tail comprises a homopolymer of A, C or T, abasic sites or modified nucleobases.

B57. The capture tag of embodiment B53, wherein the single-stranded nucleic acid tail comprises a non-self complementary, non-homopolymer sequence of more than one type of base, comprising combinations of A, C, T, G, or abasic sites which does not fold up onto itself.

B58. The capture tag of embodiment B56, wherein the single-stranded nucleic acid tail comprises a poly A homopolymer.

B59. The capture tag of any one of embodiments B52-B58, wherein the single-stranded nucleic acid tail comprises about 20 to about 1000 nucleotides.

B60. The capture tag of embodiment B59, wherein the single-stranded nucleic acid tail comprises about 40 to about 100 nucleotides.

B61. The capture tag of any one of embodiments B42-B51, wherein the single-stranded tail comprises a charged polymer.

B62. The capture tag of embodiment B61, wherein the charged polymer comprises a charged peptide polymer.

B62.1. The capture tag of embodiment B62, wherein the charged peptide polymer comprises polyarginine, polylysine or polyglutamate.

B63. The capture tag of any one of embodiments B42-B51, wherein the single-stranded tail comprises a synthetic polymer.

B64. The capture tag of embodiment B63, wherein the synthetic polymer comprises polystyrene sulfonate, polyamine, polyacrylate or polyvinyl sulfonate.

C1. A composition comprising:
a target polymer comprising monomeric units, a first distal end and a second distal end, a capture tag at the first distal end and a capture tag at the second distal end of the target polymer.

C2. The composition of embodiment C1, wherein the target polymer comprises a nucleic acid.

C2.1. The composition of embodiment C2, wherein the nucleic acid comprises greater than 250 nucleotides.

C3. The composition of embodiment C2 or C2.1, wherein the nucleic acid comprises single-stranded RNA.

C3.1. The composition of embodiment C3, wherein the single-stranded RNA comprises a plurality of short complementary DNA sequences.

C3.2. The composition of embodiment C3.1, wherein the short complementary DNA sequences comprise about 6 to about 10 nucleotides.

C4. The composition of embodiment C2 or C2.1, wherein the target polymer comprises single-stranded DNA.

C4.1. The composition of embodiment C4, wherein the single-stranded DNA comprises a plurality of annealed short complementary DNA sequences.

C4.2. The composition of embodiment C4.1, wherein the short complementary DNA sequences comprise about 6 to about 10 nucleotides.

C5. The composition of embodiment C2, wherein the target polymer comprises double-stranded DNA.

C6. The composition of embodiment C2, wherein the target polymer comprises an RNA/DNA heteroduplex.

C7. The composition of embodiment C1, wherein the target polymer comprises a protein or peptide.

C7.1. The composition of embodiment C7, wherein the protein or peptide comprises greater than 10 amino acids.

C8. The composition of any one of embodiments C1-C7.1, wherein the capture tag comprises a single-stranded tail.

C9. The composition of embodiment C8, wherein the single-stranded tail comprises nucleic acid.

C10. The composition of embodiment C9, wherein the nucleic acid comprises DNA.

C11. The composition of embodiment C9, wherein the nucleic acid comprises RNA.

C12. The composition of embodiment C10, wherein the single-stranded nucleic acid tail comprises an unstructured DNA heteropolymer or an unstructured DNA homopolymer.

C13. The composition of embodiment C10, wherein the single-stranded nucleic acid tail comprises a homopolymer of A, C or T, abasic sites or modified nucleobases.

C14. The composition of embodiment C10, wherein the single-stranded nucleic acid tail comprises a non-self-complementary, non-homopolymer sequence of more than one type of base, comprising combinations of A, C, T, G, or abasic sites which does not fold up onto itself.

C15. The composition of embodiment C13, wherein the single-stranded nucleic acid tail comprises a poly A homopolymer.

C16. The composition of any one of embodiments C9-C15, wherein the single-stranded nucleic acid tail comprises at least 20 nucleotides.

C17. The composition of embodiment C16, wherein the single-stranded nucleic acid tail comprises about 20 to about 100 nucleotides.

C18. The composition of embodiment C8, wherein the single-stranded tail comprises a charged polymer.

C19. The composition of embodiment C18, wherein the charged polymer comprises a charged peptide polymer.

C19.1. The composition of embodiment C19, wherein the charged peptide polymer comprises polyarginine, polylysine or polyglutamate.

C20. The composition of embodiment C8, wherein the single-stranded tail comprises a synthetic polymer.

C21. The composition of embodiment C20, wherein the synthetic polymer comprises polystyrene sulfonate, polyamine, polyacrylate or polyvinyl sulfonate.

C22. A composition comprising:
a single-stranded RNA target comprising a first end and a second end; and
a single-stranded tail extends from the first end and a single-stranded tail extends from the second end.

C23. A composition comprising:
single-stranded DNA target comprising a first end and a second end; and
a single-stranded tail extends from the first end and a single-stranded tail extends from the second end.

C24. A composition comprising:
a double-stranded DNA target comprising a first end, a second end, a target strand and a non-target strand; and
a single-stranded tail extends from the target strand and from the non-target strand at the first end and at the second end.

C25. The composition of embodiment C24, wherein a blocking molecule is attached to each of the single-stranded tails extending from the non-target strand of the double-stranded DNA target.

C26. The composition of embodiment C25, wherein the blocking molecule comprises biotin/streptavidin.

C27. A composition comprising:
a double-stranded DNA target comprising a first end, a second end, a target strand and a non-target strand; and
a double-stranded DNA segment comprising:
   a first end, a second end, a capture strand and a complementary strand;
   at the first end of the double-stranded DNA a single-stranded tail extends from the capture strand and a non-complementary overhang, or a single-stranded tail extends from the complementary strand or there is no extension from the complementary strand;
at the second end of the double-stranded DNA segment the capture strand is attached to the target strand of the double-stranded DNA target and the complementary strand is attached to the non-target strand of the double-stranded DNA target; and
a double-stranded DNA segment is attached at the first end and at the second end of the double-stranded DNA target.

C28. The composition of embodiment C27, wherein the double-stranded DNA segment comprises about 15 to about 1000 nucleotides.

C29. The composition of embodiment C28, wherein the double-stranded DNA segment comprises about 15 to about 20 nucleotides.

C30. The composition of any one of embodiments C27-C29, wherein a non-complementary overhang or a single-stranded tail extends from the complementary strand of the double-stranded DNA segment and the non-complementary overhang or the single-stranded tail is attached to a blocking molecule.

C31. The composition of embodiment C30, wherein the blocking molecule comprises a biotin/streptavidin complex, an antigen/antibody complex, a nanoparticle, a bulky DNA or RNA structure, a G-quadruplex, an i-motif, a cruciform-forming sequence, a pseudoknot a triple helix, a dendrimer, a polysaccharide, polyethylene glycol, a gold nanoparticle or a polystyrene nanoparticle.

C32. A composition comprising:
a single-stranded RNA target comprising a first end and a second end; and
a double-stranded DNA segment comprising:
   a first end, a second end, a capture strand and a complementary strand;
   at the first end of the double-stranded DNA segment a single-stranded tail extends from the capture strand and a non-complementary overhang or a single-stranded tail extends from the complementary strand or there is no extension from the complementary strand;
   at the second end of the double-stranded DNA segment the capture strand is attached to the single-stranded RNA target; and
a double-stranded DNA segment is attached to the first end and the second end of the single-stranded RNA target.

C33. A composition comprising:
a single-stranded DNA target comprising a first end and a second end; and
a double-stranded DNA segment comprising:
   a first end, a second end, a capture strand and a complementary strand;
   at the first end of the double-stranded DNA segment a single-stranded tail extends from the capture strand of the double-stranded DNA segment and a non-complementary overhang or a single-stranded tail extends from the complementary strand of the double-stranded DNA segment or there is no extension from the complementary strand of the double-stranded DNA segment;
   at the second end of the double-stranded DNA segment the capture strand is attached to the single-stranded DNA target; and

a double-stranded DNA segment is attached at the first end and at the second end of the single-stranded DNA target.

C34. The composition of embodiment C32 or C33, wherein the double-stranded DNA segment comprises about 15 to about 1000 nucleotides.

C35. The composition of embodiment C34, wherein the double-stranded DNA segment comprises about 15 to about 20 nucleotides.

C36. The composition of any one of embodiments C32-C35, wherein a non-complementary overhang or a single-stranded tail extends from the complementary strand of the double-stranded DNA segment and the non-complementary overhang or the single-stranded tail is attached to a blocking molecule.

C37. The composition of embodiment C36, wherein the blocking molecule comprises a biotin/streptavidin complex, an antigen/antibody complex, a nanoparticle, a bulky DNA or RNA structure, a G-quadruplex, an i-motif, a cruciform-forming sequence, a pseudoknot a triple helix, a dendrimer, a polysaccharide, polyethylene glycol, a gold nanoparticle or a polystyrene nanoparticle.

C38. A composition comprising:
an RNA/DNA heteroduplex target comprising an RNA strand, a DNA strand, a first end and a second end;
at a first end a single-stranded tails extends from the RNA strand and from the DNA strand; and
at the second end there extends a double-stranded DNA segment, wherein the double stranded DNA segment comprises:
   a first end, a second end, a capture strand and a complementary strand;
   at the first end of the double-stranded DNA segment a single-stranded tail extends from the capture strand and from the complementary strand of the double-strand; and
   at the second end of the double-stranded DNA segment the capture strand is attached to the RNA strand of the RNA/DNA duplex target and the complementary strand is attached to the DNA strand of the RNA/DNA duplex target.

C39. A composition comprising:
an RNA/DNA heteroduplex target comprising a first end, a second end, a DNA strand and an RNA strand; and
a double-stranded DNA segment comprising:
   a first end, a second end, a capture strand and a complementary strand;
   at the first end of the double-stranded DNA segment a single-stranded tail extends from the capture strand and a non-complementary overhang or a single-stranded tail extends from the complementary strand or there is no extension from the complementary strand;
   at the second end of the double-stranded DNA segment the capture strand is attached to the RNA strand of the RNA/DNA heteroduplex target and the complementary strand is attached to the DNA strand of the RNA/DNA heteroduplex target; and
a double-stranded DNA segment is attached at the first end and at the second end of the RNA/DNA heteroduplex target.

C40. The composition of embodiment C39, wherein the double-stranded DNA segment comprises about 15 to about 1000 nucleotides.

C41. The composition of embodiment C40, wherein the double-stranded DNA segment comprises about 15 to about 20 nucleotides.

C42. The composition of any one of embodiments C39-C41, wherein a non-complementary overhang or a single-stranded tail extends from the complementary strand of the double-stranded DNA segment and the non-complementary overhang or the single-stranded tail is attached to a blocking molecule.

C43. The composition of embodiment C42, wherein the blocking molecule comprises a biotin/streptavidin complex, an antigen/antibody complex, a nanoparticle, a bulky DNA or RNA structure, a G-quadruplex, an i-motif, a cruciform-forming sequence, a pseudoknot a triple helix, a dendrimer, a polysaccharide, polyethylene glycol, a gold nanoparticle or a polystyrene nanoparticle.

C44. The composition of any one of embodiments C22-C43, wherein the single-stranded tail comprises nucleic acid.

C45. The composition of embodiment C44, wherein the nucleic acid comprises DNA.

C46. The composition of embodiment C44, wherein the nucleic acid comprises RNA.

C47. The composition of embodiment C45, wherein the single-stranded nucleic acid tail comprises an unstructured DNA heteropolymer or an unstructured DNA homopolymer.

C48. The composition of embodiment C45, wherein the single-stranded nucleic acid tail comprises a homopolymer of A, C or T, abasic sites or modified nucleobases.

C49. The composition of embodiment C45, wherein the single-stranded nucleic acid tail comprises a non-self complementary, non-homopolymer sequence of more than one type of base, comprising combinations of A, C, T, G, or abasic sites which does not fold up onto itself.

C50. The composition of embodiment C48, wherein the single-stranded nucleic acid tail comprises a poly A homopolymer.

C51. The composition of any one of embodiments C44-C50, wherein the single-stranded nucleic acid tail comprises at least 20 nucleotides.

C52. The composition of embodiment C51, wherein the single-stranded nucleic acid tail comprises about 20 to about 100 nucleotides.

C53. The composition of any one of embodiments C22-C43, wherein the single-stranded tail comprises a charged polymer.

C54. The composition of embodiment C53, wherein the charged polymer comprises a charged peptide polymer.

C54.1. The composition of embodiment C54, wherein the charged peptide polymer comprises polyarginine, polylysine or polyglutamate.

C55. The composition of any one of embodiments C22-C43, wherein the single-stranded tail comprises a synthetic polymer.

C56. The composition of embodiment C55, wherein the synthetic polymer comprises polystyrene sulfonate, polyamine, polyacrylate or polyvinyl sulfonate.

C57. A composition comprising:
a protein or peptide target comprising an N terminus and a C terminus; and
a double-stranded DNA segment comprising:
   a first end, a second end, a capture strand and a complementary strand;
   at the first end of the double-stranded DNA segment a single-stranded tail extends from the capture strand and a non-complementary overhang or a single-stranded tail extends from the complementary strand or there is no extension from the complementary strand;
   at the second end of the double-stranded segment a single-stranded tail extends from the capture strand or there is no extension from the capture strand, the capture strand is configured to attach to an N terminus or a C terminus of a protein or a peptide and a non-complementary overhang or a single-stranded tail extends from the complementary strand or there is no extension from the complementary strand; and
there is a double-stranded DNA segment at the N terminus and at the C terminus of the protein or the peptide target.

C58. The composition of embodiment C57, wherein at the second end of the double-stranded DNA segment the capture strand comprises a reactive group.

C59. The composition of embodiment C57 or C58, wherein the double-stranded DNA segment at the N terminus and at the C terminus of the protein or the peptide target have the same length.

C60. The composition of embodiment C57 or C58, wherein the double-stranded DNA segment at the N terminus and at the C terminus of the protein or the peptide target have a different composition or a different length.

C61. The composition of any one of embodiments C57-C60, wherein at the first and the second end of the double-stranded DNA segment a non-complementary overhang or a single-stranded tail extends from the complementary strand and the non-complementary overhang or the single-stranded tail is attached to a blocking molecule.

C62. The composition of embodiment C61, wherein the blocking molecule comprises a biotin/streptavidin complex, an antigen/antibody complex, a nanoparticle, a bulky DNA or RNA structure, a G-quadruplex, an i-motif, a cruciform-forming sequence, a pseudoknot a triple helix, a dendrimer, a polysaccharide, polyethylene glycol, a gold nanoparticle or a polystyrene nanoparticle.

C63. The composition of any one of embodiments C57-C62, wherein the double-stranded DNA segment comprises about 1000 to about 10,000 base pairs.

C64. The composition of embodiment C63, wherein the double-stranded DNA segment comprises about 1,500 to about 5,000 base pairs.

C65. A composition comprising:
a protein or peptide target comprising an N terminus and a C terminus; and
a rigid polymer comprising
   a first end and a second end;
   a single-stranded tail extends from the first end of the rigid polymer;
   a single-stranded tail extends from the second end of the rigid polymer or there is no extension from the second end of the rigid polymer; and
   the second end of the rigid polymer is configured to attach to an N terminus or a C terminus of a protein or a peptide; and
a rigid polymer is attached at the N terminus and at the C terminus of the protein or the peptide target.

C66. The composition of embodiment C65, wherein the second end of the rigid polymer comprises a reactive group.

C67. The composition of embodiment C65 or C66, wherein the rigid polymer at the N terminus and at the C terminus of the protein or the peptide target have the same length.

C68. The composition of embodiment C65 or C66, wherein the rigid polymer at the N terminus and at the C terminus of the protein or the peptide target have a different composition and/or a different length.

C69. The composition of any one of embodiments C65-C68, wherein the rigid polymer comprises a polypeptide.

C70. The composition of embodiment C69, wherein the polypeptide is a single alpha-helix (SAH), a collagen-like helix or a coil-coil structure.

C71. The composition of embodiment C70, wherein the polypeptide is a single alpha-helix (SAH) comprising about 50 to about 3000 amino acids.

C72. The composition of any one of embodiments C57-C71, wherein the single-stranded tail comprises nucleic acid.

C73. The composition of embodiment C72, wherein the nucleic acid comprises DNA.

C74. The composition of embodiment C72, wherein the nucleic acid comprises RNA.

C75. The composition of embodiment C73, wherein the single-stranded nucleic acid tail comprises an unstructured DNA heteropolymer or an unstructured DNA homopolymer.

C76. The composition of embodiment C73, wherein the single-stranded nucleic acid tail comprises a homopolymer of A, C or T, abasic sites or comprises modified nucleobases.

C77. The composition of embodiment C73, wherein the single-stranded nucleic acid tail comprises a non-self complementary, non-homopolymer sequence of more than one type of base, comprising combinations of A, C, T, G, or abasic sites which does not fold up onto itself.

C78. The composition of embodiment C76, wherein the single-stranded nucleic acid tail comprises a poly A homopolymer.

C79. The composition of any one of embodiments C72-C78, wherein the single-stranded nucleic acid tail comprises about 20 to about 1000 nucleotides.

C80. The composition of embodiment C79, wherein the single-stranded nucleic acid tail comprises about 40 to about 100 nucleotides.

C81. The composition of any one of embodiments C57-C71, wherein the single-stranded tail comprises a charged polymer.

C82. The composition of embodiment C81, wherein the charged polymer comprises a charged peptide polymer.

C82.1. The composition of embodiment C82, wherein the charged peptide polymer comprises polyarginine, polylysine or polyglutamate.

C83. The composition of any one of embodiments C57-C71, wherein the single-stranded tail comprises a synthetic polymer.

C84. The composition of embodiment C83, wherein the synthetic polymer comprises polystyrene sulfonate, polyamine, polyacrylate or polyvinyl sulfonate.

C85. A composition comprising;
a protein or peptide target comprising an N terminus and a C terminus;
a capture tag at the N terminus and at the C terminus, wherein the capture tags comprise;
a rigid polymer comprising;
   a first end and a second end;
   a single-stranded tail extends from the first end of the rigid polymer;
   a single-stranded tail extends from the second end of the rigid polymer no extension from the second end of the rigid polymer; and
   the second end of the rigid polymer is configured to attach to an N or there is terminus or a C terminus of a protein or a peptide; or
a double-stranded DNA segment comprising:
   a first end, a second end, a capture strand and a complementary strand;
   at the first end of the double-stranded DNA segment a single-stranded tail extends from the capture strand and a non-complementary overhang or a single-stranded tail extends from the complementary strand or there is no extension from the complementary strand;
   at the second end of the double-stranded segment a single-stranded tail extends from the capture strand or there is no extension from the capture strand, the capture strand is configured to attach to an N terminus or a C terminus of a protein or a peptide and a non-complementary overhang, or a single-stranded tail extends from the complementary strand or there is no extension from the complementary strand; and
the capture tag at each end of the target polymer has a different composition and/or a different length.

D1. A method for providing a single-stranded DNA target or a single-stranded RNA target with capture tags comprising:
providing a single-stranded DNA target or a single-stranded RNA target;
providing single-stranded DNA segments comprising a single-stranded nucleic acid tail;
attaching the single-stranded DNA segments comprising a single-stranded nucleic acid tail to each end of the single-stranded DNA target or single-stranded RNA target; and
annealing a complementary oligonucleotide to each of the single-stranded DNA segments to form DNA duplexes; thereby providing a single-stranded DNA target or a single-stranded RNA target with capture tags at each end.

D2. The method of embodiment D1, wherein the single-stranded DNA segment is about 10 to about 25 nucleotides.

D3. The method of embodiment D1 or D2, wherein the single-stranded nucleic acid tails comprise a poly-A tail.

D3.1. The method of embodiment D3, wherein the poly-A tails comprise 20 or more nucleotides.

D4. The method of any one of embodiments D1-D3.1, wherein the complementary oligonucleotide comprises a non-complementary overhang adjacent to the single-stranded nucleic acid tail.

D5. The method of embodiment D4, wherein the non-complementary overhang is attached to a blocking group.

D6. The method of embodiment D5, wherein the blocking group is a biotin/streptavidin complex, an antigen/antibody complex, a nanoparticle, a bulky DNA or RNA structure, a G-quadruplex, an i-motif, a cruciform-forming sequence, a pseudoknot a triple helix, a dendrimer, a polysaccharide, polyethylene glycol, a gold nanoparticle or a polystyrene nanoparticle.

D7. A method for providing a single-stranded RNA target with capture tags comprising:
providing a single-stranded RNA target having a 3′ end and a 5′ end;
providing capture tags, wherein the capture tags are single-stranded nucleic acid tails;
enzymatically incorporating a single-stranded nucleic acid tail at the 3′ end of the single-stranded RNA target,
producing a reactive cap at the 5′ end of the single-stranded RNA target and utilizing the reactive cap to incorporate a single-stranded nucleic acid tail at the 5′ end of the single-stranded RNA target; thereby providing a single-stranded RNA target with capture tags at the 5′ end and the 3′ end.

D8. The method of embodiment D7, wherein the single-stranded nucleic acid tails comprise a poly-A tail.

D9. The method of embodiment D8, wherein the poly-A tail comprises 20 or more nucleotides.

D10. A method for providing a single-stranded RNA target with capture tags comprising:
providing a single-stranded RNA target having a 3′ end and a 5′ end;
modifying the 5′ and the 3′ ends of the single-stranded RNA target with a unique orthogonal reactive group;
providing capture tails, wherein each capture tag comprises a single-stranded tail comprising an entity that specifically reacts with the unique orthogonal reactive group on the 5′ end or the 3′ end of the single-stranded RNA target; and
chemically ligating each unique orthogonal reactive group on the 5′ end and the 3′ end of the single-stranded RNA target with the entity on the single-stranded tail that specifically reacts with the unique orthogonal reactive group; thereby producing a single-stranded RNA target with capture tags at each end.

D11. The method of embodiment D10, wherein the single-stranded tails comprise nucleic acid.

D12. The method of embodiment D11, wherein the nucleic acid comprises poly-A and the single-stranded tails are poly-A tails.

D13. The method of embodiment D12, wherein the poly-A tails comprise 20 or more nucleotides.

D14. A method for providing a single-stranded DNA target with capture tags comprising:
providing a single-stranded DNA target having a 3′ end and a 5′ end;
modifying the 5′ and the 3′ ends of the single-stranded DNA target with a unique orthogonal reactive group;
providing capture tails, wherein each capture tail comprises a single-stranded tail comprising an entity that specifically reacts with the unique orthogonal reactive group on the 5′ end or the 3′ end of the single-stranded DNA target; and
chemically ligating each unique orthogonal reactive group on the 5′ end and the 3′ end of the single-stranded DNA target with the entity on the single-stranded tail that specifically reacts with the unique orthogonal reactive group; thereby producing a single-stranded DNA target with capture tags at each end.

D15. The method of embodiment D14, wherein the single-stranded tails comprise nucleic acid.

D16. The method of embodiment D11, wherein the nucleic acid comprises poly-A and the single-stranded tails are poly-A tails.

D17. The method of embodiment D16, wherein the poly-A tails comprise 20 or more nucleotides.

D18. A method for providing a single-stranded DNA target or a single-stranded RNA target with capture tags, comprising:
providing a single-stranded DNA target or a single-stranded RNA target, each comprising a first end and a second end;
providing capture tags, wherein each capture tag comprises:
   a double-stranded DNA segment comprising:
      a first end, a second end, a capture strand and a complementary strand;
      at the first end of the double-stranded DNA segment a single-stranded tail extends from the capture strand and a non-complementary overhang or a single-stranded tail extends from the complementary strand or there is no extension from the complementary strand;
      at the second end of the double-stranded DNA segment the strand is configured for attachment to the single-capture stranded DNA target or single-stranded RNA target; and
attaching the second end of a double-stranded DNA segment to each end of the single-stranded DNA target or single-stranded RNA target; thereby providing a single-stranded DNA or single-stranded RNA target with a capture tag at each end.

D19. The method of embodiment D18, wherein attachment is by chemical ligation or enzymatic ligation.

D19.1. The method of embodiment D19, wherein the capture strand comprises a chemical functionality at the second end of the double-stranded DNA segment for covalent attachment to the single-stranded DNA target or single-stranded RNA target.

D19.2. The method of embodiment D18, wherein if present a non-complementary overhang or single stranded tail extending from the complementary strand is attached to a blocking molecule.

D20. A method for providing a double-stranded DNA target with capture tags, comprising:
providing a double-stranded DNA target comprising a first end, a second end, a target strand and a non-target strand;
providing capture tags comprising
   a double-stranded DNA segment comprising:
      a first end, a second end, a capture strand and a complementary strand;
      at the first end of the double-stranded DNA segment a single-stranded tail extends from the capture strand and a non-complementary overhang, or a single-stranded tail extends from the complementary strand or there is no extension from the complementary strand;
      at the second end of the double-stranded DNA segment the capture strand is configured for attachment to the target strand of the double-stranded DNA target and the complementary strand is configured for attachment to the non-target strand of the double-stranded DNA target; and
attaching at the first and the second end of the double-stranded DNA target the capture strand to the target strand of the double-stranded DNA target and the complementary strand to the non-target strand of the double-stranded DNA target, thereby providing a double-stranded DNA target with a capture tag at each end.

D21. The method of embodiment D20, wherein attachment is by chemical ligation or enzymatic ligation.

D21.1. The method of embodiment D21, wherein the enzymatic ligation is blunt end ligation.

D21.2. The method of embodiment D21, wherein the enzymatic ligation is sticky end ligation.

D21.3. The method of embodiment D20, wherein if present a non-complementary overhang or single stranded tail extending from the complementary strand is attached to a blocking molecule.

D22. A method for providing an RNA/DNA heteroduplex target with capture tags, comprising:
providing an RNA/DNA heteroduplex target comprising a first end, a second end, a DNA strand and an RNA strand;
providing capture tags comprising
   a double-stranded DNA segment comprising:
      a first end, a second end, a capture strand and a complementary strand;
      at the first end of the double-stranded DNA segment a single-stranded tail extends from the capture strand and a non-complementary overhang or a single-stranded tail extends from the complementary strand or there is no extension from the complementary strand;
      at the second end of the double-stranded DNA segment the capture strand is configured for attachment to the RNA strand of the RNA/DNA heteroduplex target and the complementary strand is configured for attachment to the DNA strand of the RNA/DNA heteroduplex target; and
attaching at the first and the second end of the RNA/DNA heteroduplex target the capture strand to the RNA strand of the RNA/DNA heteroduplex target and the complementary strand configured to the DNA strand of the RNA/DNA heteroduplex target, thereby providing a RNA/DNA heteroduplex target with a capture tag at each end.

D23. The method of embodiment D22, wherein attachment is by chemical ligation or enzymatic ligation.

D23.1. The method of embodiment D22, wherein if present a non-complementary overhang or single stranded tail extending from the complementary strand is attached to a blocking molecule.

D24. A method for providing an RNA/DNA heteroduplex target with capture tags, comprising:
providing an RNA/DNA heteroduplex target comprising an RNA strand, a DNA strand, a first end and a second end;
providing a capture tag comprising
   a double-stranded DNA segment, wherein the double stranded DNA comprises: segment
      a first end, a second end, a capture strand and a complementary strand;
      at the first end of the double-stranded DNA segment a single-stranded tail extends from the capture strand and a single-stranded tail extends from the complementary strand;
      at the second end of the double-stranded DNA segment the capture strand is configured for attachment to the RNA strand of the RNA/DNA duplex target and the complementary strand is configured for attachment to the DNA strand of the RNA/DNA duplex target;
attaching at the first end of the RNA/DNA heteroduplex target the capture strand of the double-stranded DNA segment to the RNA strand of the RNA/DNA heteroduplex target and the complementary strand of the double-stranded DNA segment to the DNA strand of the RNA/DNA heteroduplex target; and
attaching at the second end of the RNA/DNA heteroduplex target single-stranded nucleic acid tails by ligation of a single-stranded nucleic acid tail to the DNA strand of the RNA/DNA heteroduplex target and by synthesis of a single-stranded nucleic acid tail on the RNA strand of the RNA/DNA heteroduplex target, thereby providing an RNA/DNA heteroduplex target with capture tags at each end.

D25. A method for providing a double-stranded DNA target with capture tags, comprising:
providing a double-stranded DNA target comprising a first end, a second end, a target strand and a non-target strand;
chemically modifying the target strand of the double-stranded DNA target to provide a reactive group at each end;
chemically modifying the non-target strand of the double-stranded DNA target to provide a reactive group at each end, wherein the reactive group is different from the reactive group on the target strand;
chemically ligating a single-stranded nucleic acid tail to each end of a target strand of the double-stranded DNA target; and
chemically ligating a single-stranded nucleic acid tail attached to a blocking molecule to each end of a non-target strand of the double-stranded DNA target; thereby providing a double-stranded DNA target with capture tags at each end.

D26. A method for providing a protein or peptide target with a capture tag, comprising:
providing a protein or peptide target comprising an N terminus and a C terminus;
providing a capture tag comprising a double-stranded DNA segment wherein the double stranded DNA segment comprises:
   a first end, a second end, a capture strand and a complementary strand;
   at the first end of the double-stranded DNA segment a single-stranded tail extends from the capture strand and a non-complementary overhang or a single-stranded tail extends from the complementary strand or there is no extension from the complementary strand;
   at the second end of the double-stranded segment a single-stranded tail extends from the capture strand or there is no extension from the capture strand, the capture strand is modified to comprise a reactive group to attach to an N terminus or a C terminus of a protein or a peptide and a non-complementary overhang or a single-stranded tail extends from the complementary strand or there is no extension from the complementary strand; and
attaching the capture strand of the double-stranded segment to the N terminus and/or the C terminus of the target protein or a peptide; thereby providing a protein or peptide target with a capture tag.

D26.1. The method of embodiment D26, wherein if present a non-complementary overhang or single stranded tail extending from the complementary strand is attached to a blocking molecule.

D27. A method for providing a protein or peptide target with a capture tag, comprising:
providing a protein or peptide target comprising an N terminus and a C terminus; and
providing a capture tag comprising;
   a rigid polymer comprising a first end and a second end;
   a single-stranded tail extending from the first end of the rigid polymer;
   a single-stranded tail extending from the second end of the rigid polymer or there is no extension from the second end of the rigid polymer;
   the second end of the rigid polymer is modified to comprise a reactive group to attach to an N terminus or a C terminus of a protein or a peptide; and attaching the second end of the rigid to the N terminus and/or the C terminus of the protein or the peptide target, thereby providing a protein or peptide target with a capture tag.

E1. A method of sequencing a target polymer comprising:
providing a tagged target polymer comprising any one the compositions of embodiments C1-C85;
providing a dual nanopore device; and
contacting the tagged target polymer and the dual nanopore device; and
identifying monomeric units of the tagged target polymer as the tagged polymer translocates through the first nanopore or the second nanopore, thereby determining the sequence of at least a portion of the target polymer.

F1. A system comprising:
a dual nanopore device; and
a tagged target polymer comprising any one the compositions of embodiments C1-C85.

F2. The system of embodiment F1, wherein a dual nanopore device comprises:
a chip;
a first well disposed adjacent to a second well on the chip;
a first seal over the first well and a second seal over the second well, and
a first nanopore is in the first seal and a second nanopore is in the second seal.

F3. The system of embodiment F1 or F2, wherein each nanopore independently is tethered to one or more magnetic particles.

F4. The system of embodiments F2 or F3, wherein each nanopore comprises a biological nanopore.

G1. A method of associating each of a plurality of target polymers with a dual nanopore device comprising:
providing a plurality of tagged target polymers each comprising any one the compositions of embodiments C1-C85;
providing a plurality of dual nanopore devices; and
contacting the plurality of tagged target polymers and the plurality of dual nanopore devices.

H1. A system comprising:
a plurality of dual nanopore devices; and
a plurality of tagged target polymers each comprising any one the compositions of embodiments C1-C85.

H2. The system of embodiment H1, wherein a dual nanopore device comprises:
a chip;
a first well disposed adjacent to a second well on the chip;
a first seal over the first well and a second seal over the second well, and
a first nanopore is in the first seal and a second nanopore is in the second seal.

H3. The system of embodiment H1 or H2, wherein each nanopore independently is tethered to one or more magnetic particles.

H4. The system of embodiment H2 or H3, wherein each nanopore comprises a biological nanopore.

H5. The system of any one of embodiments H1-H4, wherein each of the plurality of dual nanopore devices is independently controlled.

11. A method of sequencing a plurality of target polymers comprising:
providing a plurality of tagged target polymers each comprising any one the compositions of embodiments C1-C85;
providing a plurality of dual nanopore devices;
contacting the plurality of tagged target polymers and the plurality of dual nanopore devices; and
identifying monomeric units of the tagged target polymers as each of the tagged target polymers translocate through a dual nanopore device.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (for example, “a reagent” can mean one or more reagents) unless it is contextually clear either one of the elements or more than one of the elements is described. The term “about” as used herein refers to a value within 10% of the underlying parameter (i.e., plus or minus 10%; for example, a weight of “about 100 grams” can include a weight between 90 grams and 110 grams). Use of the term “about” at the beginning of a listing of values modifies each of the values (for example, “about 1, 2 and 3” refers to “about 1, about 2 and about 3”). When a listing of values is described the listing includes all intermediate values and all fractional values thereof (for example, the listing of values “80%, 85% or 90%” includes the intermediate value 86% and the fractional value 86.4%). When a listing of values is followed by the term “or more,” the term “or more” applies to each of the values listed (for example, the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (for example, the listing of “80%, 90% or 95%” includes ranges of “80% to 90%,” “80% to 95%” and “90% to 95%”).

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Certain implementations of the technology are set forth in the claims that follow. A number of embodiments of the invention have been described. Nevertheless, it can be understood that various modifications may be made without departing from the spirit and scope of the invention. Accordingly, other embodiments are within the scope of the following claims.

1. A method for associating, joining or linking a target polymer with dual nanopores, comprising:
(a) providing a target polymer comprising monomeric units, a first distal end, a second distal end, a capture tag at the first distal end and a capture tag at the second distal end, thereby providing a tagged polymer;
(b) providing a first nanopore and a second nanopore;
(c) driving the first distal end of the tagged polymer through the first nanopore, thereby capturing the first distal end of the tagged polymer by the first nanopore; and
(d) driving the second distal end of the tagged polymer through the second nanopore, thereby capturing the second distal end of the tagged polymer by the second nanopore.

2. The method of claim 1, wherein a capture tag comprises a single-stranded tail.

3. The method of claim 2, wherein the single-stranded tail comprises nucleic acid.

4. The method of claim 3, wherein the single-stranded nucleic acid tail comprises a poly A homopolymer.

5. The method of claim 2, wherein the single-stranded nucleic acid tail comprises at least 20 nucleotides.

6. The method of claim 1, wherein the first nanopore and the second nanopore are part of a dual nanopore device.

7. The method of claim 1, wherein each nanopore comprises a biological nanopore.

8. tagged polymer through the first nanopore and/or driving the second distal end of the tagged polymer through the second nanopore comprises electrophoretic control and/or electroosmotic control and/or pressure driven flow.

9. The method of claim 1, wherein after step (d),
electrophoretically and/or electroosmotically driving at least a portion of the tagged polymer through the first nanopore or at least a portion of the tagged polymer through the second nanopore; and
identifying monomeric units of the tagged polymer as the tagged polymer translocates through the first nanopore or the second nanopore, thereby determining the sequence of at least a portion of the target polymer.

10. The method of claim 1, wherein:
(e) after step (d), electrophoretically and/or electroosmotically driving at least a portion of the tagged polymer through the first nanopore;
(f) the method further comprises identifying monomeric units of the tagged polymer as the tagged polymer translocates through the first nanopore, thereby determining the sequence of at least a portion of the target polymer;
(g) the method further comprises reversing the voltage bias between the first nanopore and the second nanopore;
(h) the method further comprises electrophoretically and/or electroosmotically driving at least a portion of the tagged polymer through the second nanopore;
(i) the method further comprises identifying monomeric units of the tagged polymer as the tagged polymer translocates through the second nanopore, thereby determining the sequence of at least a portion of the target polymer;
(j) the method further comprises reversing the voltage bias between the first nanopore and the second nanopore;
(k) the method further comprises electrophoretically and/or electroosmotically driving at least a portion of the tagged polymer through the first nanopore;
(l) the method further comprises identifying monomeric units of the tagged polymer as the tagged polymer translocates through the first nanopore, thereby determining the sequence of at least a portion of the target polymer; or
(m) the method further comprises repeating steps (g) to (l) to re-read the sequence or portion of the sequence of the target polymer.

11. The method of claim 1, wherein the target polymer comprises a single stranded RNA, a single-stranded DNA, a double-stranded DNA, an RNA/DNA heteroduplex, a protein, or a peptide.

12. A system, comprising:
a dual nanopore device; and
a tagged target polymer comprising a capture tag at a first distal end and a capture tag at a second distal end, wherein the capture tag comprises a single-stranded tail.

13. The system of claim 12, wherein the target polymer comprises single-stranded RNA, single-stranded DNA, double-stranded DNA, an RNA/DNA heteroduplex, a protein, or a peptide.

14. A composition, comprising a target polymer comprising monomeric units, a first distal end and a second distal end, a capture tag at the first distal end and a capture tag at the second distal end of the target polymer, wherein the capture tag comprises a single-stranded tail.

15. The composition of claim 14, wherein the target polymer comprises single-stranded RNA, single-stranded DNA, double-stranded DNA, an RNA/DNA heteroduplex, a protein, or a peptide.

16. A capture tag, comprising a double-stranded DNA segment comprising a first end, a second end, a capture strand and a complementary strand, wherein:
at the first end of the double-stranded DNA segment a single-stranded tail extends from the capture strand and an overhang or a single-stranded tail extends from the complementary strand, or there is no extension from the complementary strand; and
at the second end of the double-stranded DNA segment the capture strand or the capture strand and the complementary strand are configured to attach to a target polymer.

17. The capture tag of claim 16, wherein a non-complementary overhang or a single-stranded tail extends from the complementary strand of the double-stranded DNA segment and the non-complementary overhang or the single-stranded tail is attached to a blocking molecule.

18. A capture tag, comprising: a double-stranded DNA segment comprising a capture strand with a single-stranded tail attached at one end of the capture strand and an opposite end of the capture strand configured to attach to a N-terminus or a C-terminus of a target protein or peptide.

19. A method for providing a target polymer with capture tags comprising:
providing a target polymer;
providing capture tags comprising a single-stranded tail; and
attaching a capture tag to each end of the target polymer.

20. The method of claim 19, wherein the target polymer comprises single-stranded RNA, single-stranded DNA, double-stranded DNA, an RNA/DNA heteroduplex, a protein, or a peptide.

21. A kit, comprising or having contained therein a system of claim 12.
